(19) United States
(12) Patent Application Publication
(10) Pub. No.: US 2025/0194641 A1
(43) Pub. Date: Jun. 19, 2025

(54) Materials and Methods for Iron Capture and Greenhouse Gas Reduction

(71) Applicant: Locus Solutions IPCo, LLC, Solon, OH (US)

(72) Inventor: Maja MILOVANOVIC, North Royalton, OH (US)

(21) Appl. No.: 18/843,824

(22) PCT Filed: Jul. 7, 2023
(86) PCT No.: PCT/US2023/069754

§ 371 Date: Sep. 4, 2024

Related U.S. Application Data
(60) Provisional application No. 63/359,248, filed on Jul. 8, 2022.

(51) Int. Cl. A23K 10/18; A23K 10/30; A23K 20/10; A23K 20/22; A23K 50/10; A61K 35/74

(52) U.S. Cl. CPC A23K 10/18; A23K 10/30; A23K 20/10; A23K 20/22; A23K 50/10; A61K 35/74

(57) ABSTRACT

The subject invention provides compositions and methods for reducing enteric methane in livestock animals' digestive systems and/or waste. In preferred embodiments, a composition comprising an iron-capturing ingredient is contacted with the livestock animal's digestive system and/or waste in order to, for example, limit iron availability to methanogenic microorganisms and thus reduce methanogenesis.

### CROSS-REFERENCE TO RELATED APPLICATION

This application claims priority to U.S. Provisional Application No. 63/359,248, filed Jul. 8, 2022, which is incorporated herein by reference in its entirety.

### BACKGROUND OF THE INVENTION

Gases that trap heat in the atmosphere are called “greenhouse gases,” or “GHG,” and include carbon dioxide, methane, nitrous oxide and fluorinated gases (EPA report 2016 at 6).

Carbon dioxide (CO₂) enters the atmosphere through burning fossil fuels (coal, natural gas, and oil), solid waste, trees and wood products, and also as a result of certain chemical reactions, e.g., the manufacture of cement. Carbon dioxide is removed from the atmosphere by, for example, absorption by plants as part of the biological carbon cycle.

Nitrous oxide (N₂O) is emitted during industrial activities and during combustion of fossil fuels and solid waste. In agriculture, over-application of nitrogen-containing fertilizers and poor soil management practices can also lead to increased emissions of nitrous oxide and other nitrogen-based gases.

Fluorinated gases including, e.g., hydrofluorocarbons, perfluorocarbons, sulfur hexafluoride, and nitrogen trifluoride are synthetic, powerful greenhouse gases that are emitted from a variety of industrial processes.

Methane (CH4) is emitted during the production and transport of coal, natural gas, and oil. Furthermore, other agricultural practices, and the decay of organic waste in lagoons and municipal solid waste landfills can produce methane emissions. Notably, however, methane emissions also result from production of livestock animals, many of whose digestive systems comprise methanogenic microorganisms (Overview of Greenhouse Gases 2016).

Based on recent measurements from monitoring stations around the world and measurement of older air from air bubbles trapped in layers of ice from Antarctica and Greenland, global atmospheric concentrations of GHGs have risen significantly over the last few hundred years (EPA report 2016 at, e.g., 6, 15).

Especially since the Industrial Revolution began in the 1700s, human activity has contributed to the amount of GHGs in the atmosphere by burning fossil fuels, cutting down forests, and conducting other activities. Many GHGs emitted into the atmosphere remain there for long periods of time ranging from a decade to many millennia. Over time these gases are removed from the atmosphere by chemical reactions or by emissions sinks, such as the oceans and vegetation that absorb GHGs from the atmosphere.

World leaders have attempted to curb the increase of GHG emissions through treaties and other inter-state agreements. One such attempt is through the use of carbon credit systems. A carbon credit is a generic term for a tradable certificate or permit representing the right to emit one ton of carbon dioxide, or an equivalent GHG. In a typical carbon credit system, a governing body sets quotas on the amount of GHG emissions an operator can produce. Exceeding these quotas requires the operator to purchase extra allowances from other operators who have not used all of their carbon credits.

One goal of carbon credit systems is to encourage companies to invest in more green technology, machinery and practices in order to benefit from the trade of these credits. Under the Kyoto Protocol of the United Nations Framework Convention on Climate Change (UNFCCC), a large number of countries have agreed to be bound internationally by policies for GHG reduction, including through trade of emissions credits. While the United States is not bound by the Kyoto Protocol, and while there is no central national emissions trading system in the U.S., some states, such as California and a group of northeastern states, have begun to adopt such trading schemes.

Another attempt to reduce atmospheric GHGs, in particular, methane emissions, has involved the use of feed additives or supplements in livestock production. Ruminant livestock, such as, for example, cattle, sheep, buffalo, goats, deer and camels, are unique because of their four stomach compartments: the reticulum, rumen, omasum and abomasum. The rumen, in particular, is a large, hollow organ where microbial fermentation of ingested substances, such as fibrous plant material, occurs. This organ can hold 40-60 gallons of material, with an estimated microbial concentration of 150 billion microbes per teaspoon of rumen contents.

The rumen functions as a microaerophilic fermentation vessel for certain bacteria that produce gaseous fermentation by-products, including oxygen, nitrogen, H₂ and carbon dioxide. Methanogenesis is a natural process contributing to the efficiency of the digestive system, reducing the partial pressure of H₂ and allowing the normal functioning of microbial enzymes. The process is regulated by methanogens, the most common of which is Methanobrevibacter. Methanogens form a biofilm on surfaces where hydrogen-producing bacteria and protozoa actively produce H₂ required for reducing carbon dioxide to methane.

As an example, cattle, raised for both beef and milk, as well as for inedible outputs like manure and draft power, are responsible for the greatest amounts of emissions from livestock, representing about 65% of the livestock sector's emissions. Approximately 130 to over 250 gallons of ruminal gas produced by fermentation can be belched from one cow each day. This is important for the health of the cow, as it prevents bloating; however, the negative result is the emission of GHG such as carbon dioxide and methane into the atmosphere.

Other animals, including non-ruminant animals, also contribute to enteric GHG production. For example, swine, rodents, monkeys, horses, mules, asses, rhinoceros, hippopotamuses, bears, poultry, and certain other birds also contain methanogenic bacteria in their digestive systems. Certain monogastric animals also produce N₂O and CO₂ emissions.

In addition to gut fermentation, livestock manure can also be a source of GHG emissions.

Manure contains two components that can lead to GHG emissions during storage and processing: organic matter that can be converted into methane emissions, and nitrogen that leads indirectly to nitrous oxide emissions. Methane is released when methanogenic bacteria decompose the organic material in the manure as it is being held in lagoons, tailing ponds or holding tanks. Additionally, nitrogen in the form of ammonia (NH₃) is released from manure and urine during storage and processing. Ammonia can later be transformed into nitrous oxide. (Gerber et al. 2013).

Currently, approaches for reducing livestock methane emissions include defaunation of the digestive system and even vaccination against methanogens. The downsides to these strategies, however, are that they may reduce the number of beneficial gut microbes, and the methods may be short-lived due to microbial adaptation. Additionally, energy providers have attempted to harvest methane from manure lagoons and collection ponds as a form of biogas fuel; however, the methods are inefficient and do not capture significant amounts of methane relative to the total amount of methane produced by livestock production.

Other strategies have involved dietary modification, particularly for livestock grazing pasture, in order to manipulate gut fermentation by, for example, directly inhibiting methanogens and protozoa, or by redirecting hydrogen ions away from the methanogens to reduce methanogenesis. Such dietary modifications include, for example, the addition of probiotics, acetogens, bacteriocins, ionophores (e.g., monensin and lasalocid), organic acids and/or plant extracts (e.g., tannins and/or seaweed), to feed. (Ishler 2016). Most anti-methanogenic compounds are costly, short-lived, show inconsistent results, require high concentrations, do not contain H₂ acceptors, do not affect methanogens in the form of biofilms, and comprise compounds that are easily destroyed and/or excreted.

Specific feed additive products include, for example, Mootral, Bovaer®, and FutureFeed, each of which has its own limitations. Mootral is a feed supplement comprising a proprietary combination of active compounds from garlic, and flavonoids derived from citrus. The product works by direct inhibition/killing of methanogenic bacteria. The amount of actual methane reduction, about 30%, is low however, compared to the cost per cow per year. Additionally, the product can increase the urea content of milk, indicating an increase in nitrous oxide production.

Bovaer® is a feed additive comprising 3-nitrooxy propanol, which inhibits methyl coenzyme M reductase, an enzyme that catalyzes the final step in methanogenesis. The amount of actual methane reduction achieved is also relatively low, at about 30%, and relatively short-term in duration. Additionally, due to a lack of H₂ acceptors, the product can cause a fishy smell in produced milk because of an increase in trimethylamine production.

FutureFeed is a feed supplement that utilizes a specific type of seaweed that can reduce enteric methane emissions by up to 98%. Nonetheless, the product is costly at $200 per kg and is slow to have a consistent impact on enteric gas production.

The livestock industry is important for the production of, for example, meats and dairy products; however, growing concerns over climate change and a need for reducing GHG emissions calls for improved approaches for producing livestock with reduced GHG emissions.

### BRIEF SUMMARY OF THE INVENTION

The subject invention provides compositions and methods for reducing atmospheric greenhouse gas emissions from livestock animals. More specifically, the subject invention provides compositions that, when contacted with the digestive system and/or waste of a livestock animal, lead to a reduction in greenhouse gas (GHG) emissions that would have otherwise been produced by the animal's digestive processes and/or waste. Advantageously, the compositions and methods can also enhance the overall health of a livestock animal.

In preferred embodiments, the subject invention provides methods for reducing enteric methane and/or other deleterious atmospheric gases and/or precursors thereof in a livestock animal's digestive system or waste, wherein a composition comprising an iron-capturing ingredient is introduced into the animal's digestive system. The composition can be administered enterally and/or parenterally.

Advantageously, in preferred embodiments, the methods result in a direct inhibition of methanogenic bacteria, increased out-competition of methanogens by beneficial microorganisms, disruption of methanogenic biofilms, and/or disruption of the biological pathway involved in methanogenesis in the livestock animal's digestion system, for example, the rumen, stomach and/or intestines.

More specifically, in certain embodiments, the methods result in capture of iron present in the livestock animal's digestive system, thereby inhibiting methanogenesis via iron limitation. In preferred embodiments, the iron is sequestered in such a way that it can be made available to the animal's digestive system, while being unavailable to methanogenic microorganisms therein.

The subject invention further provides a composition comprising one or more ingredients that capture iron. In certain embodiments, the iron-capturing ingredient is a beneficial microorganism, a growth by-product of a microorganism, or some other compound known to bind iron. In certain embodiments, more than one iron-capturing ingredient is included in the composition.

In preferred embodiments, the beneficial microorganisms of the subject invention are non-pathogenic fungi, yeasts and/or bacteria capable of sequestering iron, either naturally or through genetic modification.

In one embodiment, the beneficial microorganism is a strain of Bacillus subtilis. In a specific embodiment, the strain is B. subtilis B4 (NRRL B-68031). The B4 strain is preferably administered in spore form but grows in biofilm form when exposed to acidic environments within the digestive system.

Surprisingly, B4 was found to produce one or more compounds capable of sequestering, chelating or otherwise capturing iron. In certain embodiments, the compounds are pulcherrimin and/or pulcherriminic acid. Advantageously, the microbes and/or the exopolysaccharide (EPS) of the microbes when grown in biofilm form effectively hoard freely-available iron using an iron-capturer such as pulcherrimin and/or pulcherriminic acid while traveling through acidic methanogen inhabited portions of the ruminant digestive system. In some embodiments, the iron is made available to the digestive system as a nutrient upon exposure to a more neutral pH.

B4 is also particularly advantageous over other traditional probiotic microorganisms due to its ability to produce digestive enzymes, including, for example, cellulases and amylases.

In certain embodiments, the composition can comprise other non-pathogenic microorganisms that are capable of producing compounds that can sequester, chelate or otherwise capture iron. The microorganism(s) can be in biofilm form, spore form, planktonic form, or any other form.

In certain embodiments, the microorganisms are also capable of producing one or more of the following: surface active agents, such as lipopeptides and/or glycolipids; bioactive compounds with antimicrobial and immune-modulating effects; polyketides; acids; peptides; anti-inflammatory compounds; enzymes, such as amylases, cellulases, proteases and/or lipases; and sources of amino acids, vitamins, and other nutrients.

In certain embodiments, the iron-capturing ingredient of the subject composition is a crude form or purified siderophore or phytosiderophore, or other molecule with high iron affinity, for example, pulcherrimin, pulcherriminic acid, citrate, citric acid, EDTA (Ethylenediaminetetraacetic acid), ferric EDTA, DTPA (Diethylenetriaminepentaacetic acid), EDDHA (Ethylenediamine di(o-hydroxyphenylacetic acid), N,N-dihydroxy-N,N′-diisopropylhexanediamide (DPH), 2,3-dihydroxybenzoic acid, azotochelin, transferrin, enterobactin, pyoverdine, protochelin, pyochelin, bacillibactin, vibriobactin, vibrioferrin azotobactin, aminochelin, yersiniabactin, agrobactin, staphyloferrin, ferrichrome, defarasirox, deferiprone, desferrioxamine, fusarinine, chrysobactin, achromobactin, ornibactin, rhodotorulic acid, lysine, glutamic acid, gluconic acid, iron oxyhydroxide minerals, ferrihydrite, magnetite, hematite, geothite, sideritehydroxamate, catecholates, salicylates, carboxylates, mugineic acid, ferulic acid, caffeic acid, and/or nicotianamine.

In certain embodiments, the composition comprises one or more enzymes that help digest food sources into smaller units, such as volatile fatty acids (e.g., propionate, acetate, butyrate), glucose and amino acids.

In certain embodiments, the composition can be formulated for enteral and/or parenteral administration to the livestock animal's digestive system. For example, in certain embodiments, the composition can be formulated as dry feed pellets, powders and/or granules to supplement grains and/or forage (e.g., pasture plants, hay, silage and/or crop residue), while in others, the composition can be administered as an injectable formulation.

In certain embodiments, the methods and compositions of the subject invention also reduce GHG emissions from the livestock animal's waste (e.g., urine and/or manure). In some embodiments, the beneficial microorganisms of the composition can survive transport through the digestive system and are excreted with the animal's waste, where they continue inhibiting methanogens and/or symbionts thereof, disrupting methanogenic biofilms, disrupting the biological pathways involved in methanogenesis, and/or compensating for H₂ acceptor loss. The composition can be administered to the livestock animal's digestive system and/or directly to the waste product.

In certain embodiments, the methods and compositions of the subject invention decrease methanogenesis while also increasing glucose and milk synthesis. An increase in digestive enzymes by administration of the subject composition to an animal's digestive system can lead to an increase in propionate production, which effectively regulates the pH and robs methanogens of the required components for methanogenesis. In general, ruminant fermentation results in the release of negatively charged ions into the digestive system environment, increasing the acidity of the environment. These negatively charged ions are required for methanogenesis, so a highly acidic environment encourages methanogen growth; however, the synthesis of propionate also requires negatively charged ions.

In certain embodiments, the increase in propionate production achieved through the administration of the subject composition also supports milk production in nursing animals. Propionate is absorbed through the portal vein of the liver, wherein it is used to create glucose within the liver, which supports milk synthesis.

The methods can further comprise adding materials to enhance the growth of the microorganisms of the composition at the time of application (e.g., adding nutrients and/prebiotics). In certain embodiments, the livestock animal can be fed a source of prebiotics, which can include, for example, dry animal fodder, straw, hay, alfalfa, grains, forage, grass, fruits, vegetables, oats, and/or crop residue.

In some embodiments, the methods can be utilized for enhancing the overall health of a livestock animal, for example, by contributing to a healthy gut microbiome, improving digestion, increasing feed-to-muscle conversion ratio, increasing milk production and quality, reducing and/or treating dehydration and heat stress, modulating the immune system, and increasing life expectancy.

In some embodiments, the methods of the subject invention can be utilized by a livestock producer for reducing carbon credit usage. Thus, in certain embodiments, the subject methods can further comprise conducting measurements to assess the effect of the method on reducing the generation of methane, carbon dioxide and/or other deleterious atmospheric gases, and/or precursors thereof (e.g., nitrogen and/or ammonia), and/or to assess the effect of the method on the control of methanogens in the livestock animal's digestive system and/or waste, using standard techniques in the art.

### BRIEF DESCRIPTION OF THE FIGURES

FIG. 1 shows total mean methane concentration (ml/g of feed) measured for two different dosage treatments of the B4 strain compared with untreated control (UTC) according to an embodiment of the subject invention.

FIG. 2 shows mean methane concentration (ml/g of feed) measured at 24 hours, 48 hours, and 72 hours for two different dosage treatments of the B4 strain compared with untreated control (UTC) according to an embodiment of the subject invention.

FIGS. 3A-3C show growth and exopolysaccharide (EPS) formation of B4 after 24 hours (A) and 48 hours (B) at acidic pH (top row, pH 4.8) and neutral pH (bottom row, pH 6.8).

FIG. 4 shows purified B4 EPS with a pink hue.

FIG. 5 shows result of an amylase test for B4. Agar streaked with B4 produced an orange color around the bacterial growth, indicating the breakdown of starch.

FIG. 6 show results of a cellulase test for B4. Agar streaked with B4 produced a yellow zone of clearing around the bacterial growth, indicating the breakdown of cellulose.

FIGS. 7A-7C show B4 siderophore production and activity after 6 hours in aerobic (top plates) and anaerobic (bottom plates) environments and on different growth media. (A) shows B4 culture grown in MRS-sucrose (left side) and M23-6 (right side) media. (B) shows B4 culture grown in minimal media with Tween (left side) and minimal media without Tween (right side). (C) shows dried B4 spores grown in minimal medium.

FIGS. 8A-8C show B4 siderophore production and activity after 24 hours in aerobic (top plates) and anaerobic (bottom plates) environments and on different growth media. (A) shows B4 culture grown in M23-6 (left side) and MRS-sucrose (right side) media. (B) shows B4 culture grown in minimal media with Tween (left side) and minimal media without Tween (right side). (C) shows dried B4 spores grown in minimal medium.

FIG. 9 shows results of an iron assay for B4 cultures grown in different media.

### DETAILED DESCRIPTION OF THE INVENTION

The subject invention provides compositions and methods for reducing atmospheric greenhouse gas emissions from livestock production. More specifically, the subject invention provides compositions that, when contacted with the digestive system and/or waste of a livestock animal, lead to a reduction in greenhouse gas emissions that would have otherwise been produced by the animal's digestive processes. Advantageously, in some embodiments, the compositions and methods can also improve the overall health and productivity of livestock animals.

In specific embodiments, the subject invention provides a composition for reducing methane and/or other deleterious atmospheric gases and/or precursors thereof produced in a livestock animal's digestive system and/or waste, wherein the composition comprises one or more ingredients capable of capturing freely-available iron in the digestive system.

### Selected Definitions

As used herein, a “biofilm” is a complex aggregate of microorganisms, such as bacteria, wherein the cells adhere to each other and/or to a surface. In certain embodiments, adherence is achieved via an exopolysaccharide substance produced by the bacteria. The cells in biofilms are physiologically distinct from planktonic cells of the same organism, which are single cells that can float or swim in liquid medium.

As used herein, the term “control” used in reference to an undesirable microorganism (e.g., a methanogen) extends to the act of killing, disabling, immobilizing and/or reducing the population numbers of the microorganism, and/or otherwise rendering the microorganism incapable of reproducing and/or carrying out the processes that are undesirable (e.g., methane production).

As used herein, the “digestive system” refers to the system of organs in an animal's body that enables digestion, or the consumption of food and conversion thereof to energy and waste. The digestive system can comprise, for example, an oral cavity, esophagus, crop, gizzard, proventriculus, stomach, rumen, reticulum, omasum, abomasum, pancreas, liver, small intestine, large intestine (colon), cecum, appendix, and/or anus. Additional organs or parts related to digestion and that are specific to a particular animal are also envisioned.

As used herein, an “isolated” or “purified” nucleic acid molecule, polynucleotide, polypeptide, protein, organic compound such as a small molecule (e.g., those described below), or other compound is substantially free of other compounds, such as cellular material, with which it is associated in nature. For example, a purified or isolated polynucleotide (ribonucleic acid (RNA) or deoxyribonucleic acid (DNA)) is free of the genes or sequences that flank it in its naturally-occurring state. A purified or isolated polypeptide is free of the amino acids or sequences that flank it in its naturally-occurring state. A purified or isolated microbial strain is removed from the environment in which it exists in nature. Thus, the isolated strain may exist as, for example, a biologically pure culture, or as spores (or other forms of the strain) in association with a carrier.

In certain embodiments, purified compounds are at least 60% by weight the compound of interest. Preferably, the preparation is at least 75%, more preferably at least 90%, and most preferably at least 99%, by weight the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis.

As used herein, “ionophores” are carboxylic polyether non-therapeutic antibiotics that disrupt the ion concentration gradient (Ca2+, K+, H+, Na+) across microorganisms, which causes them to enter a futile ion cycle. The disruption of the ion concentration prevents the microorganism from maintaining normal metabolism and causes the microorganism to expend extra energy.

Ionophores function by selecting against or affecting the metabolism of gram-positive bacteria, such as methanogens, and protozoa.

As used herein, “siderophores” are compounds produced by different organisms for the purpose of scavenging iron from the surrounding environment. Siderophores are typically small, low molecular weight compounds with high affinity for ferric iron (Fe³⁺), forming strong ferric chelate complexes that can, in some instances be taken up by the organisms. As used herein, “phytosiderophores” are siderophores produced by plants.

A “metabolite” refers to any substance produced by metabolism (e.g., a growth by-product) or a substance necessary for taking part in a particular metabolic process. A metabolite can be an organic compound that is a starting material, an intermediate in, or an end product of metabolism. Examples of metabolites can include, but are not limited to, enzymes, toxins, acids, solvents, alcohols, proteins, carbohydrates, vitamins, minerals, microelements, amino acids, polymers, polyketides, and surfactants.

As used herein, a “methanogen” is a microorganism that produces methane gas as a by-product of metabolism. Methanogens are archaea that can be found in the digestive systems and metabolic waste of ruminant animals and non-ruminant animals (e.g., pigs, poultry and horses). Examples of methanogens include, but are not limited to, Methanobacterium spp. (e.g., M. formicicum), Methanobrevibacter spp. (e.g., M. ruminantium), Methanococcus spp. (e.g., M. paripaludis), Methanoculleus spp. (e.g., M. bourgensis), Methanoforens spp. (e.g., M. stordalenmirensis), Methanofollis liminatans, Methanogenium wolfei, Methanomicrobium spp. (e.g., M. mobile), Methanopyrus kandleri, Methanoregula boonei, Methanosaeta spp. (e.g., M. concilii, M. thermophile), Methanosarcina spp. (e.g., M. barkeri, M. mazeii), Methanosphaera stadtmanae, Methanospirillium hungatei, Methanothermobacter spp., and/or Methanothrix sochngenii.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

As used herein, “reduction” means a negative alteration and “increase” means a positive alteration, wherein the positive or negative alteration is at least 0.25%, 0.5%, 1%, 5%, 10%, 15%, 20%, 25%, 30%, 35%, 40%, 45%, 50%, 55%, 60%, 65%, 70%, 75%, 80%, 85%, 90%, 95% or 100%.

The transitional term “comprising,” which is synonymous with “including,” or “containing,” is inclusive or open-ended and does not exclude additional, un-recited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention. Use of the term “comprising” contemplates other embodiments that “consist” or “consist essentially of” the recited component(s).

Unless specifically stated or obvious from context, as used herein, the term “or” is understood to be inclusive. Unless specifically stated or obvious from context, as used herein, the terms “a,” “and” and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

All references cited herein are hereby incorporated by reference in their entirety.

### Compositions

In preferred embodiments, the subject invention provides methods for reducing enteric methane and/or other deleterious atmospheric gases and/or precursors thereof in a livestock animal's digestive system or waste, wherein a composition comprising an iron-capturing ingredient is introduced into the animal's digestive system. The composition can be administered enterally and/or parenterally.

The subject invention further provides a composition for use according to the subject methods, wherein the composition comprises one or more ingredients that capture iron. In certain embodiments, the iron-capturing ingredient is a beneficial microorganism, a growth by-product of an iron-capturing microorganism, or some other compound known to bind iron. In certain embodiments, more than one iron-capturing ingredient is included in the composition.

The total iron-capturing ingredient(s) in the composition preferably comprise from 0.0001% to 100% of the composition by weight or by volume, or from 0.001 to 95%, from 0.01 to 90%, from 0.1% to 85%, from 0.5 to 80%, from 0.75 to 75%, from 1.0 to 70%, from 1.25 to 65%, from 1.5 to 60%, from 1.75 to 55%, from 2.0 to 50%, or from 5.0 to 25% by weight or by volume.

In certain embodiments, the digestive health composition is a “microbe-based composition,” meaning a composition that comprises components that were produced as the result of the growth of microorganisms or other cell cultures. Thus, the microbe-based composition may comprise the microbes themselves and/or by-products of microbial growth. The microbes may be in a vegetative state, in spore form, in mycelial form, in any other form of microbial propagule, or a mixture of these. The microbes may be planktonic or in a biofilm form, or a mixture of both. The by-products of growth may be, for example, metabolites, cell membrane components, expressed proteins, and/or other cellular components. The microbes may be intact or lysed. The cells may be totally absent, or present at, for example, a concentration of at least 1×10⁴, 1×10⁵, 1×10⁶, 1×10⁷, 1×10⁸, 1×10⁹, 1×10¹⁰, 1×10¹¹, 1×10¹², 1×10¹³ or more CFU per milliliter or CFU/g of the composition.

Advantageously, in preferred embodiments, the subject compositions can alter the digestive processes of livestock animals, resulting in decreased enteric atmospheric gas production.

In some embodiments, the subject compositions can be used for reducing methane, carbon dioxide and/or nitrous oxide production in livestock and/or livestock waste. For example, the compositions can directly inhibit or control methanogenic bacteria and/or symbionts thereof in the animal's digestive system and/or waste, as well as disrupt the integrity and/or production of biofilms formed by methanogens. Additionally, in some embodiments, the compositions can interfere with biological pathways involved in methanogenesis.

In some embodiments, the composition can also enhance the growth and health of livestock, while enabling more complete transformation of protein sources in feed to reduce nitrogen release in the animals' waste in the form of, e.g., ammonia and/or urea. Advantageously, in some embodiments, this can result in reduced nitrous oxide production.

In preferred embodiments, the beneficial microorganisms of the subject compositions are non-pathogenic fungi, yeasts and/or bacteria capable of sequestering iron, either naturally or through genetic modification. The beneficial microorganisms may be in an active, inactive and/or dormant form. In preferred embodiments, the microorganism is one that is characterized as “generally regarded as safe,” or GRAS, by the appropriate regulatory agency.

In certain embodiments, the microorganism is pathogenic, but utilized only in the production of growth by-products as opposed to direct administration to an animal.

In certain embodiments, the microorganisms are also capable of producing one or more of the following: surface active agents, such as lipopeptides and/or glycolipids; bioactive compounds with antimicrobial and immune-modulating effects; polyketides; acids; peptides; anti-inflammatory compounds; enzymes, such as amylases, cellulases, proteases and/or lipases; and sources of amino acids, vitamins, and other nutrients.

The microorganisms of the subject invention may be natural, or genetically modified microorganisms. For example, the microorganisms may be transformed with specific genes to exhibit specific characteristics. The microorganisms may also be mutants of a desired strain. As used herein, “mutant” means a strain, genetic variant or subtype of a reference microorganism, wherein the mutant has one or more genetic variations (e.g., a point mutation, missense mutation, nonsense mutation, deletion, duplication, frameshift mutation or repeat expansion) as compared to the reference microorganism. Procedures for making mutants are well known in the microbiological art. For example, UV mutagenesis and nitrosoguanidine are used extensively toward this end.

In some embodiments, the beneficial microorganisms are selected based on a natural or acquired resistance to certain antibiotics administered to a livestock animal to, for example, control pathogenic and/or deleterious microbes in the digestive system or elsewhere in the animal's body.

In some embodiments, the beneficial microorganisms of the subject composition are capable of surviving transport through the livestock animal's digestive system and are excreted in the animal's waste (e.g., manure). Thus, in certain embodiments, administering a composition according to embodiments of the subject invention to the animal can result in a reduction in GHG production in the animal's waste via inhibition of methanogens and/or symbionts thereof, disruption of methanogen biofilms, interference with biological pathways involved in methanogenesis, and compensation for H₂ acceptor loss.

In one specific embodiment, the composition comprises about 1×10⁶ to about 1×10¹³, about 1×10⁷ to about 1×10¹², about 1×10⁸ to about 1×10¹¹, or about 1×10⁹ to about 1×10¹⁰ CFU/g of each species of microorganism present in the composition.

In one embodiment, the composition comprises about 1 to 100% microorganisms total by volume, about 10 to 90%, or about 20 to 75%. In one embodiment, the composition comprises microbial growth by-products but no living microorganisms.

In an exemplary embodiment, the amount of microorganisms in one application of the composition totals about 1 to 100 grams per head (individual animals in a herd or flock), or about 5 to about 85 grams per head, or about 10 to about 70 grams per head, or about 15 to 50 grams per head.

In one embodiment, the microorganisms are yeasts, bacteria and/or fungi, including, for example, Acaulospora, Acidithiobacillus spp. (e.g., A. ferooxidans, A. albertensis, A. caldus, A. cuprithermicus, A. ferrianus, A. ferridurans, A. ferriphilus, A. ferrivorans, A. ferrooxidans, A. sulfuriphilus, and A. thiooxidans), Acremonium chrysogenum, Agrobacterium (e.g., A. radiobacter), Aspergillus, Aureobasidium (e.g., A. pullulans), Azospirillum (e.g., A. brasiliensis), Azotobacter (A. vinelandii, A. chroococcum), Bacillus (e.g., B. amyloliquefaciens, B. coagulans, B. firmus, B. laterosporus, B. licheniformis, B. megaterium, B. mucilaginosus, B. subtilis), Blakeslea, Candida (e.g., C. albicans, C. apicola, C. batistae, C. bombicola, C. floricola, C. kuoi, C. riodocensis, C. nodaensis, C. stellate), Cryptococcus, Debaryomyces (e.g., D. hansenii), Dipodascopsism, Entomophthora, Escherichia coli, Frateuria (e.g., F. aurantia), Hanseniaspora (e.g., H. uvarum), Hansenula, Issatchenkia, Kluyveromyces (e.g., K. phaffii), Lentinula spp. (e.g., L. edodes), Legionella pneumophila, Lipomyces, Magnetospirillum magneticum, Magnetococcus marinus, Metschnikowia sp. (M. pulcherrimia), Meyerozyma (e.g., M. guilliermondii, M. caribbica), Monascus purpureus, Mortierella, Mucor (e.g., M. piriformis), Neisseria meningitidis, Pantoea (e.g., P. agglomerans, P. allii), Penicillium, Phythium, Phycomyces, Pichia (e.g., P. anomala, P. guilliermondii, P. occidentalis, P. kudriavzevii), Pleurotus (e.g., P. ostreatus P. ostreatus, P. sajorcaju, P. cystidiosus, P. cornucopiae, P. pulmonarius, P. tuberregium, P. citrinopileatus and P. flabellatus), Pseudomonas (e.g., P. chlororaphis, P. aeruginosa, P. koreensis), Pseudozyma (e.g., P. aphidis, P. antarctica), Rhizobium radiobacter, Rhizopus, Rhodospirillum (e.g., R. rubrum), Rhodotorula (e.g., R. bogoriensis), Saccharomyces (e.g., S. cerevisiae, S. boulardii, S. torula), Sphingomonas (e.g., S. paucimobilis), Starmerella (e.g., S. bombicola), Streptomyces, Torulopsis, Thraustochytrium, Trichoderma (e.g., T. reesei, T. harzianum, T. viridae), Ustilago (e.g., U. maydis), Vibrio cholerae, Wickerhamiella (e.g., W. domericqiae), Wickerhamomyces (e.g., W. anomalus), Williopsis (e.g., W. mrakii), Zygosaccharomyces (e.g., Z. bailii), and others.

Fungi can be in the form of live or inactive cells, mycelia, spores and/or fruiting bodies. The fruiting bodies, if present, can be, for example, chopped and/or blended into granules and/or a powder form.

Yeasts can be in the form of live or inactive cells or spores, as well as in the form of dried and/or dormant cells (e.g., a yeast hydrolysate).

Bacteria can be in the form of vegetative or planktonic cells, biofilms, spores, and/or a dried cell or spore mass.

In some embodiments, dried microbes, e.g., spores, can be mixed with fillers known in the art, such as e.g., microcrystalline cellulose (MCC).

In one embodiment, the composition comprises one or more Bacillus spp. bacteria and/or growth by-products thereof. In certain embodiments, the Bacillus spp. are B. amyloliquefaciens, B. subtilis, B. coagulans and/or B. licheniformis.

In one embodiment, the composition comprises B. amyloliquefaciens NRRL B-67928 “B. amy” and/or a growth by-product thereof. A culture of the B. amyloliquefaciens “B. amy” microbe has been deposited with the Agricultural Research Service Northern Regional Research Laboratory (NRRL) Culture Collection, 1815 N. University St., Peoria, IL, USA. The deposit has been assigned accession number NRRL B-67928 by the depository and was deposited on Feb. 26, 2020.

In one embodiment, the composition comprises a strain of Bacillus subtilis and/or a growth by-product thereof. In a specific embodiment, the strain is B. subtilis B4 (NRRL B-68031). A culture of the B4 microbe has been deposited with the Agricultural Research Service Northern Regional Research Laboratory (NRRL) Culture Collection, 1815 N. University St., Peoria, IL, USA. The deposit has been assigned accession number NRRL B-68031 by the depository and was deposited on May 6, 2021.

B4 is a Gram-positive spore-forming strain of B. subtilis that is capable of anaerobic growth (obligate anaerobe). The B4 strain is preferably administered in spore form but germinates in the livestock animal's gut, wherein it can grow in biofilm form when exposed to acidic environments within the digestive system. Surprisingly, B4 was found to produce one or more compounds capable of sequestering, chelating or otherwise capturing iron when grown in biofilm form. In certain embodiments, the compounds are pulcherrimin and/or pulcherriminic acid.

Advantageously, the microbes and/or the exopolysaccharide (EPS) of the biofilm effectively hoard freely-available iron using an iron-capturer such as, e.g., pulcherrimin and/or pulcherriminic acid, while traveling through low pH (e.g., less than 5) methanogen inhabited portions of the ruminant digestive system. In some embodiments, the iron is made available to the digestive system as a nutrient upon exposure to a more neutral pH, e.g., 5 to 7.

B4 is also particularly advantageous over other traditional probiotic microorganisms due to its ability to produce increased amounts of the lipopeptide surfactin (e.g., greater than wild type B. subtilis), and its ability to produce digestive enzymes, including, for example, cellulases and amylases. These enzymes help digest food sources into smaller units, such as volatile fatty acids (e.g., propionate, acetate, butyrate), glucose and amino acids. In certain embodiments, increased propionate synthesis decreases hydrogen concentrations, which decreases methanogenesis, while also increasing glucose and milk synthesis.

The proprietary cultures described herein have been deposited under conditions that assure that access to the cultures will be available during the pendency of this patent application to one determined by the Commissioner of Patents and Trademarks to be entitled thereto under 37 CFR § 1.14 and 35 U.S.C § 122. The deposit is available as required by foreign patent laws in countries wherein counterparts of the subject application, or its progeny, are filed. However, it should be understood that the availability of a deposit does not constitute a license to practice the subject invention in derogation of patent rights granted by governmental action.

Further, each of the subject culture deposits will be stored and made available to the public in accord with the provisions of the Budapest Treaty for the Deposit of Microorganisms, i.e., it will be stored with all the care necessary to keep it viable and uncontaminated for a period of at least five years after the most recent request for the furnishing of a sample of the deposit, and in any case, for a period of at least 30 (thirty) years after the date of deposit or for the enforceable life of any patent which may issue disclosing the culture. The depositor acknowledges the duty to replace the deposit should the depository be unable to furnish a sample when requested, due to the condition of the deposit. All restrictions on the availability to the public of the subject culture deposit will be irrevocably removed upon the granting of a patent disclosing it.

In certain embodiments, the composition can comprise other microorganisms that are capable, either naturally or by genetic modification, of producing pulcherrimin and/or pulcherriminic acid, or other compounds capable of sequestering, chelating or otherwise capturing iron. In specific embodiments, the microbes are capable of growing as a biofilm.

In certain embodiments, the microorganism is a naturally-occurring or genetically-modified microorganism capable of regulating genes involved in iron capture and transport, e.g., HFE, GDF15, TWSG1, ERFE, Matriptase 2, TF, TFR1, TFR2, HAMP and HJV.

In certain embodiments, the iron-capturing ingredient of the subject composition is a crude form or purified siderophore or phytosiderophore, or other molecule with high iron affinity, for example, pulcherrimin, pulcherriminic acid, citrate, citric acid, EDTA (Ethylenediaminetetraacetic acid), ferric EDTA, DTPA (Diethylenetriaminepentaacetic acid), EDDHA (Ethylenediamine di(o-hydroxyphenylacetic acid), N,N-dihydroxy-N,N′-diisopropylhexanediamide (DPH), 2,3-dihydroxybenzoic acid, azotochelin, transferrin, enterobactin, pyoverdine, protochelin, pyochelin, bacillibactin, vibriobactin, vibrioferrin azotobactin, aminochelin, yersiniabactin, agrobactin, staphyloferrin, ferrichrome, defarasirox, deferiprone, desferrioxamine, fusarinine, chrysobactin, achromobactin, ornibactin, rhodotorulic acid, lysine, glutamic acid, gluconic acid, iron oxyhydroxide minerals, ferrihydrite, magnetite, hematite, geothite, sideritehydroxamate, catecholates, salicylates, carboxylates, mugineic acid, ferulic acid, caffeic acid, and/or nicotianamine.

The composition can also comprise other microbial growth by-products. The microbial growth by-product can be produced by the microorganisms of the composition, and/or they can be produced separately, e.g., by a microorganism listed herein, and added to the composition.

In one embodiment, the growth by-product has been purified from the cultivation medium in which it was produced. Alternatively, in one embodiment, the growth by-product is utilized in crude form. The crude form can comprise, for example, a liquid supernatant resulting from cultivation of a microbe that produces the growth by-product of interest, including residual cells and/or nutrients.

The growth by-products can include metabolites or other biochemicals produced as a result of cell growth, including, for example, amino acids, peptides, polyketides, antibiotics, proteins, enzymes, biosurfactants, solvents, vitamins, and/or other metabolites.

The microorganism(s) and/or growth by-product(s) present in the composition can be useful for inhibiting methanogens and/or the methanogenesis pathway, disrupting methanogen biofilms, and/or reducing H₂ accumulation in a livestock animal's digestive system. Furthermore, in preferred embodiments, the composition can be useful for enhancing the overall health of a livestock animal.

### Additional Components

In certain embodiments, the composition comprises a germination enhancer for enhancing germination of spore-form microorganisms used in the microbe-based composition. In specific embodiments, the germination enhancers are amino acids, such as, for example, L-alanine and/or L-leucine. In one embodiment, the germination enhancer is manganese.

In one embodiment, the composition comprises one or more fatty acids. The fatty acids can be produced by the microorganisms of the composition, and/or produced separately and included as an additional component. In certain preferred embodiments, the fatty acid is a saturated long-chain fatty acid, having a carbon backbone of 14-20 carbons, such as, for example, myristic acid, palmitic acid or stearic acid. In some embodiments, a combination of two or more saturated long-chain fatty acids is included in the composition. In some embodiments, a saturated long-chain fatty acid can inhibit methanogenesis and/or increase cell membrane permeability of methanogens.

In certain embodiments, the composition comprises one or more enzymes that help digest food sources into smaller units, such as volatile fatty acids (e.g., propionate, acetate, butyrate), glucose and amino acids. These enzymes can be produced by the microorganisms of the composition, and/or produced separately and included as an additional component Non-limiting examples of digestive enzymes include amylases, maltases, lactases, lipases, proteases, sucrases and cellulases.

In certain embodiments, the composition comprises a chelating agent including, but are not limited to, ethylenediaminetetraacetic acid (EDTA), nitrilotriacetic acid (NTA), a phosphonate, succimer (DMSA), diethylenetriaminepentaacetate (DTPA), N-acetylcysteine, n-hydroxyethylethylenediaminetriacetic acid (HEDTA), organic acids with more than one coordination group (e.g., rubeanic acid), STPP (sodiumtripolyphosphate, Na5P3O10), trisodium phosphate (TSP), water, carbohydrates, organic acids with more than one coordination group (e.g., citric acid), lipids, steroids, amino acids or related compounds (e.g., glutathione), peptides, phosphates, nucleotides, tetrapyrrols, ferrioxamines, ionophores, orphenolics, sodium citrate, sodium gluconate, ethylenediamine disuccinic acid (EDDS), iminodisuccinic acid (IDS), L-glutamic acid diacetic Acid (GLDA), GLDA-Na₄, methyl glycindiacetic acid (MGDA), polyaspartic acid (PASA), hemoglobin, chlorophyll, lipophilic β-diketone, and (14,16)-hentriacontanedione, ethylenediamine-N,N′-diglutaric acid (EDDG), ethylenediamine-N,N′-dimalonic acid (EDDM), 3-hydroxy-2,2-iminodisuccinic acid (HIDS), 2-hydroxyethyliminodiacetic acid (HEIDA), pyridine-2,6-dicarboxylic acid (PDA), trimethyl glycine (TMG), Tiron, or any combination thereof.

In some embodiments, the composition can comprise additional components known to reduce methane in the livestock animal's digestive system, such as, for example, nitrates; seaweed (e.g., Asparagopsis taxiformis and/or Asparagopsis armata); kelp; nitrooxypropanols (e.g., 3-nitrooxypropanol and/or ethyl-3-nitrooxypropanol); anthraquinones; ionophores (e.g., monensin and/or lasalocid); polyphenols (e.g., saponins, tannins); Yucca schidigera extract (steroidal saponin-producing plant species); Quillaja saponaria extract (triterpenoid saponin-producing plant species); organosulfurs (e.g., garlic extract); flavonoids (e.g., quercetin, rutin, kaempferol, naringin, and anthocyanidins; bioflavonoids from green citrus fruits, rose hips and black currants); carboxylic acid; and/or terpenes (e.g., d-limonene, pinene and citrus extracts).

In a specific exemplary embodiment, the composition comprises 3-nitrooxypropanol (3NOP), an organic compound having the formula HOCH₂CH₂CH₂ONO₂. 3NOP is effective for suppressing one or more enzymes involved in methanogenesis, e.g., methyl coenzyme M reductase (Mcr). Mcr mediates the final step of all methanogenesis pathways, with CoM (2-mercaptoethanesulfonic acid) as an essential co-factor serving as the methyl group carrier. Mcr reduces methyl-CoM to methane. 3NOP can competitively bind to the Mcr active site and then oxidize the Ni¹⁺ that is required for Mcr activity. (Patra et al. 2017). In some embodiments, including 3NOP in the subject composition can result in inactivation or inhibition of Mcr and thus, reduced methane emissions from livestock.

In a specific exemplary embodiment, the composition comprises nitric acid and/or nitrate salts, such as calcium nitrate, ammonium nitrate, sodium nitrate, potassium nitrate, and magnesium nitrate. Nitrates may lower methane production by competing with methanogenic bacteria for available hydrogen.

In one embodiment, the subject composition can comprise one or more additional substances and/or nutrients to supplement the livestock animal's nutritional needs and promote health and/or well-being in the livestock animal, such as, for example, sources of amino acids (including essential amino acids), peptides, proteins, vitamins, microelements, fats, fatty acids, lipids, carbohydrates, sterols, enzymes, and minerals such as calcium, magnesium, phosphorus, potassium, sodium, chlorine, sulfur, chromium, cobalt, copper, iodine, iron, manganese, molybdenum, nickel, selenium, and zinc. In some embodiments, the microorganisms of the composition produce and/or provide these substances.

In one embodiment, the composition can comprise one or more biosurfactants. Biosurfactants are a structurally diverse group of surface-active substances produced by microorganisms, which are biodegradable and can be efficiently produced using selected organisms on renewable substrates. All biosurfactants are amphiphiles. They consist of two parts: a polar (hydrophilic) moiety and non-polar (hydrophobic) group. The common lipophilic moiety of a biosurfactant molecule is the hydrocarbon chain of a fatty acid, whereas the hydrophilic part is formed by ester or alcohol groups of neutral lipids, by a carboxylate group of fatty acids or amino acids (or peptides), an organic acid in the case of flavolipids, or, in the case of glycolipids, by a carbohydrate.

Due to their amphiphilic structure, biosurfactants increase the surface area of hydrophobic water-insoluble substances, increase the water bioavailability of such substances, and change the properties of bacterial cell surfaces. Biosurfactants accumulate at interfaces, thus reducing interfacial tension and leading to the formation of aggregated micellar structures in solution. Safe, effective microbial biosurfactants reduce the surface and interfacial tensions between the molecules of liquids, solids, and gases. The ability of biosurfactants to form pores and destabilize biological membranes permits their use as antibacterial, antifungal, and hemolytic agents.

Biosurfactants according to the subject invention can include, for example, glycolipids, lipopeptides, flavolipids, phospholipids, fatty acid esters, and high molecular weight polymers such as lipoproteins, lipopolysaccharide-protein complexes, and polysaccharide-protein-fatty acid complexes.

In one embodiment, the biosurfactant is a glycolipid. Glycolipids can include, for example, sophorolipids, rhamnolipids, cellobiose lipids, mannosylerythritol lipids and trehalose lipids. In one embodiment, the biosurfactant is a lipopeptide. Lipopeptides can include, for example, surfactin, iturin, arthrofactin, viscosin, fengycin, and lichenysin. In certain embodiments, a mixture of biosurfactants is used.

In one embodiment, the biosurfactant has been purified from the fermentation medium in which it was produced. Alternatively, in one embodiment, the biosurfactant is utilized in crude form comprising fermentation broth resulting from cultivation of a biosurfactant-producing microbe. This crude form biosurfactant solution can comprise from about 0.001% to 99%, from about 25% to about 75%, from about 30% to about 70%, from about 35% to about 65%, from about 40% to about 60%, from about 45% to about 55%, or about 50% pure biosurfactant, along with residual cells and/or nutrients.

In one embodiment, the composition comprises a saponin at 1 to 10 ml/L, or 2 to 6 ml/L of ruminal fluid. Saponins are natural surfactants that are found in many plants and that exhibit similar characteristics to microbial biosurfactants, for example, self-association and interaction with biological membranes. There are three basic categories of saponins, including triterpenoid saponins, steroidal saponins, and steroidal glycoalkaloids.

Some well-known triterpenoid saponin-accumulating plant families include the Leguminosae, Amaranthaceae, Apiaceae, Caryophyllaceae, Aquifoliaceae, Araliaceae, Cucurbitaceae, Berberidaceae, Chenopodiaceae, Myrsinaceae and Zygophyllaceae, among many others. Quillaja and legumes such as soybeans, beans and peas are a rich source of triterpenoid saponins. The steroidal saponins are typically found in members of the Agavaceae, Alliaceae, Asparagaceae, Dioscoreaceae, Liliaceae, Amaryllidaceae, Bromeliaceae, Palmae and Scrophulariaceae families and accumulate in abundance in crop plants such as yam, alliums, asparagus, fenugreek, yucca and ginseng. The steroidal glycoalkaloids are commonly found in members of the Solanaceae family including tomato, potato, aubergines and capsicum.

In certain embodiments, a saponin-containing plant extract may reduce methane production by altering rumen pH and/or reducing protozal methanogen symbionts.

In one embodiment, the composition can further comprise water. For example, the microorganism and/or growth by-products can be mixed with water and administered to the livestock animal. In another embodiment, the composition can be mixed with a livestock animal's drinking water as, for example, a feed additive and/or supplement. The drinking water composition can comprise, for example, 1 g/L to about 50 g/L of the microbe-based composition, about 2 g/L to about 20 g/L, or about 5 g/L to about 10 g/L.

Advantageously, in certain embodiments, the composition can enhance hydration and reduce the occurrence and/or severity of heat stress in livestock animals.

The composition can be formulated for enteral and/or parenteral delivery to the livestock animal's digestive system. For example, the composition can be formulated for oral administration via feed, water, and/or endoscopy; and/or for administration via direct injection into one or more parts of the digestive system (e.g., the rumen, stomach and/or intestines), via endoscopy, via enema, via fecal transplant, and/or via suppository.

In certain embodiments, the composition can further comprise one or more carriers and/or excipients suitable for delivery of the composition to the digestive system of the livestock animal, preferably, to the rumen, and can be formulated into preparations in, for example, solid, semi-solid, liquid or gaseous forms, such as tablets, capsules, pressed pellets, powders, granules, ointments, gels, lotions, solutions, suppositories, drops, patches, injections, inhalants and aerosols.

Carriers and/or excipients according the subject invention can include any and all solvents, diluents, buffers (such as, e.g., neutral buffered saline, phosphate buffered saline, or optionally Tris-HCl, acetate or phosphate buffers), oil-in-water or water-in-oil emulsions, aqueous compositions with or without inclusion of organic co-solvents suitable for, e.g., IV use, solubilisers (such as, e.g., Tween 80, Polysorbate 80), colloids, dispersion media, vehicles, fillers, chelating agents (such as, e.g., EDTA or glutathione), amino acids (such as, e.g., glycine), proteins, disintegrants, binders, lubricants, wetting agents, emulsifiers, sweeteners, colorants, flavorings, aromatisers, thickeners, coatings, preservatives (such as, e.g., Thimerosal, benzyl alcohol), antioxidants (such as, e.g., ascorbic acid, sodium metabisulfite), tonicity controlling agents, absorption delaying agents, adjuvants, bulking agents (such as, e.g., lactose, mannitol) and the like. The use of carriers and/or excipients in the field of drugs and supplements is well known. Except for any conventional media or agent that is incompatible with the components of the subject compositions, its use in the subject compositions may be contemplated.

In one exemplary embodiment, the microbe-based composition can be formulated for direct administration into the digestive system or a part thereof via, for example, injection and/or endoscopy, for example, as a solution or suspension. The solution or suspension can comprise suitable non-toxic, enterally-acceptable diluents or solvents, such as mannitol, 1,3-butanediol, water, Ringer's solution or isotonic sodium chloride solution, or suitable dispersing or wetting and suspending agents, such as sterile, bland, fixed oils, including synthetic mono- or diglycerides, and fatty acids, including oleic acid. Water or saline solutions and aqueous dextrose and glycerol solutions may be preferably employed as carriers, particularly for enterally-injectable solutions.

In one exemplary embodiment, the microbe-based composition can be formulated for oral administration as a pre-made wet or dry feed, wherein the pre-made food has been cooked and/or processed to be ready for animal consumption. For example, the microorganism and/or growth by-products can be poured onto and/or mixed with the pre-made food, or the microorganism and/or growth by-products can serve as a coating on the outside of dry animal food pieces, e.g., morsels, kibbles or pellets.

In one embodiment, the composition can further comprise raw ingredients for making animal feed, wherein the raw ingredients, together with the microorganism and/or growth by-products, are then cooked and/or processed to make an enhanced dry or wet feed product. Raw ingredients can include, for example, grains, grasses, roughage, forage, hay, straw, seeds, nuts, crop residue, vegetables, fruits, dried plant matter, and other flavorings, additives and/or sources of nutrients. In one embodiment, the composition is added to the raw food ingredients at a concentration of about 0.1% to about 50%, about 1% to about 25%, or about 5% to about 15% by weight.

The microbe-based composition can be added to the wet or day feed and/or raw feed ingredients at a concentration of, for example, about 0.1% to 99%, about 1% to about 75%, or about 5% to about 50% by weight.

As used herein, “dry food” refers to food that contains a limited moisture content, typically in the range of about 5% to about 15% or 20% w/v. Typically, dry processed food comes in the form of small to medium sized individual pieces, e.g., morsels, kibbles, treats, biscuits, nuts, cakes or pellets.

The supplemented dry food pieces can comprise consistent concentrations of the microbe-based composition per piece. In another embodiment, the composition can be utilized as a surface coating on the dry food pieces. Methods known in the art for producing dry processed foods can be used, including pressurized milling, extrusion, and/or pelleting.

In an exemplary embodiment, dry food may be prepared by, e.g., screw extrusion, which includes cooking, shaping and cutting raw ingredients into a specific shape and size in a very short period of time. The ingredients may be mixed into homogenous expandable dough and cooked in an extruder, and forced through a die under pressure and high heat. After cooking, the pellets are then allowed to cool, before optionally being sprayed with a coating. This coating may comprise, for example, liquid fat or digest, including liquid or powdered hydrolyzed forms of an animal tissue such as liver or intestine from, e.g., chicken or rabbit, and/or a nutritional oil. In other embodiments, the pellet is coated using a vacuum enrobing technique, wherein the pellet is subjected to vacuum and then exposed to coating materials after which the release of the vacuum drives the coating materials inside the pellet. Hot air drying can then be employed to reduce the total moisture content to 10% or less.

In one embodiment, the dry food is produced using a “cold” pelleting process, or a process that does not use high heat or steam. The process can use, for example, liquid binders with viscous and cohesive properties to hold the ingredients together without risk of denaturing or degrading important components and/or nutrients in the compositions of the subject invention.

In one embodiment, the composition can be applied to animal fodder, or cut and dried plant matter, such as hay, straw, silage, sprouted grains, legumes and/or grains.

In one embodiment, the composition may be prepared as a spray-dried biomass product. The biomass may be separated by known methods, such as centrifugation, filtration, separation, decanting, a combination of separation and decanting, ultrafiltration or microfiltration.

In one embodiment, the composition has a high nutritional content, for example, comprising up to 50% protein, as well as polysaccharides, vitamins, and minerals. As a result, the composition may be used as part of all of a complete animal feed composition. In one embodiment, the feed composition comprises the subject composition ranging from 15% of the feed to 99% of the feed.

In one embodiment, the subject composition can comprise additional nutrients to supplement an animal's diet and/or promote health and/or well-being in the animal, such as, for example, sources of amino acids (including essential amino acids), peptides, proteins, vitamins, microelements, fats, fatty acids, lipids, carbohydrates, sterols, enzymes, prebiotics, and minerals. In some embodiments, the microorganisms of the composition produce and/or provide these substances.

Preferred compositions comprise vitamins and/or minerals in any combination. Vitamins for use in a composition of this invention can include for example, vitamins A, E, K3, D3, B1, B3, B6, B12, C, biotin, folic acid, panthothenic acid, nicotinic acid, choline chloride, inositol and para-amino-benzoic acid. Minerals can include, for example, such as calcium, magnesium, phosphorus, potassium, sodium, chlorine, sulfur, chromium, cobalt, copper, iodine, iron, manganese, molybdenum, nickel, selenium, and zinc. Other components may include, but are not limited to, antioxidants, beta-glucans, bile salt, cholesterol, enzymes, carotenoids, and many others. Typical vitamins and minerals are those, for example, recommended for daily consumption and in the recommended daily amount (RDA), although precise amounts can vary. The composition would preferably include a complex of the RDA vitamins, minerals and trace minerals as well as those nutrients that have no established RDA, but have a beneficial role in healthy mammal physiology.

### Exemplary Embodiments

In some embodiments, the composition of the subject invention comprises:
A) one or more microorganisms (yeasts, fungi and/or bacteria) capable of capturing iron and/or producing an iron-capturing growth by-product, wherein preferably at least one of the one or more microorganisms is a Bacillus sp., and wherein even more preferably, the Bacillus sp. is B. subtilis NRRL B-68031 or B. amyloliquefaciens NRRL B-67928;
B) an iron-capturing substance selected from pulcherrimin, pulcherriminic acid, citrate, citric acid, EDTA (Ethylenediaminetetraacetic acid), ferric EDTA, DTPA (Diethylenetriaminepentaacetic acid), EDDHA (Ethylenediamine di(o-hydroxyphenylacetic acid), N,N-dihydroxy-N,N′-diisopropylhexanediamide (DPH), 2,3-dihydroxybenzoic acid, azotochelin, transferrin, enterobactin, pyoverdine, protochelin, pyochelin, bacillibactin, vibriobactin, vibrioferrin azotobactin, aminochelin, yersiniabactin, agrobactin, staphyloferrin, ferrichrome, defarasirox, deferiprone, desferrioxamine, fusarinine, chrysobactin, achromobactin, ornibactin, rhodotorulic acid, lysine, glutamic acid, gluconic acid, iron oxyhydroxide minerals, ferrihydrite, magnetite, hematite, geothite, sideritehydroxamate, catecholates, salicylates, carboxylates, mugineic acid, ferulic acid, caffeic acid, and/or nicotianamine;
C) a digestive enzyme selected from amylases, maltases, lactases, lipases, proteases, sucrases and cellulases;
D) a methane-reducing substance selected from nitrates (and/or nitric acid); seaweed (e.g., Asparagopsis taxiformis and/or Asparagopsis armata); kelp; nitrooxypropanols (e.g., 3-nitrooxypropanol and/or ethyl-3-nitrooxypropanol); anthraquinones; ionophores (e.g., monensin and/or lasalocid); polyphenols (e.g., saponins, tannins); Yucca schidigera extract (steroidal saponin-producing plant species); Quillaja saponaria extract (triterpenoid saponin-producing plant species); organosulfurs (e.g., garlic extract); flavonoids (e.g., quercetin, rutin, kaempferol, naringin, and anthocyanidins; bioflavonoids from green citrus fruits, rose hips and black currants); carboxylic acid; and/or terpenes (e.g., d-limonene, pinene and citrus extracts);
E) a biosurfactant selected from sophorolipids, rhamnolipids, cellobiose lipids, mannosylerythritol lipids, trehalose lipids, surfactin, iturin, arthrofactin, viscosin, fengycin, and lichenysin; and/or
F) a nutrient and/or prebiotic.

In some embodiments, the composition comprises each of components A-F. In some embodiments, the composition comprises any combination of A-F, or any one of A-F individually.

In some embodiments, the composition comprises spore, vegetative and/or biofilm-form B. subtilis NRRL B-68031 or B. amyloliquefaciens NRRL B-67928; and pulcherrimin and/or pulcherriminic acid.

In some embodiments, the composition comprises spore, vegetative and/or biofilm-form B. subtilis NRRL B-68031 or B. amyloliquefaciens NRRL B-67928; and a nitrate, such as calcium nitrate, ammonium nitrate, potassium nitrate, magnesium nitrate and sodium nitrate.

In some embodiments, the composition comprises spore, vegetative and/or biofilm-form B. subtilis NRRL B-68031 or B. amyloliquefaciens NRRL B-67928; and a digestive enzyme, such as an amylase, maltase, lactase, lipase, protease, sucrase and/or cellulase.

In some embodiments, the composition comprises spore, vegetative and/or biofilm-form B. subtilis NRRL B-68031 or B. amyloliquefaciens NRRL B-67928; pulcherrimin and/or pulcherriminic acid; and a digestive enzyme, such as an amylase, maltase, lactase, lipase, protease, sucrase and/or cellulase.

In some embodiments, the composition comprises spore, vegetative and/or biofilm-form B. subtilis NRRL B-68031 or B. amyloliquefaciens NRRL B-67928; pulcherrimin and/or pulcherriminic acid; a digestive enzyme, such as an amylase, maltase, lactase, lipase, protease, sucrase and/or cellulase; and a nitrate, such as calcium nitrate, ammonium nitrate, potassium nitrate, magnesium nitrate and sodium nitrate.

Production of Microorganisms and/or Microbial Growth by-Products

The subject invention utilizes methods for cultivation of microorganisms and production of microbial metabolites and/or other by-products of microbial growth. The subject invention further utilizes cultivation processes that are suitable for cultivation of microorganisms and production of microbial metabolites on a desired scale. These cultivation processes include, but are not limited to, submerged cultivation/fermentation, solid state fermentation (SSF), and modifications, hybrids and/or combinations thereof.

As used herein “fermentation” refers to cultivation or growth of cells under controlled conditions. The growth could be aerobic or anaerobic. In preferred embodiments, the microorganisms are grown using SSF and/or modified versions thereof.

In one embodiment, the subject invention provides materials and methods for the production of biomass (e.g., viable cellular material), extracellular metabolites, residual nutrients and/or intracellular components.

The microbe growth vessel used according to the subject invention can be any fermenter or cultivation reactor for industrial use. In one embodiment, the vessel may have functional controls/sensors or may be connected to functional controls/sensors to measure important factors in the cultivation process, such as pH, oxygen, pressure, temperature, humidity, microbial density and/or metabolite concentration.

In a further embodiment, the vessel may also be able to monitor the growth of microorganisms inside the vessel (e.g., measurement of cell number and growth phases).

Alternatively, a daily sample may be taken from the vessel and subjected to enumeration by techniques known in the art, such as dilution plating technique.

In one embodiment, the method includes supplementing the cultivation with a nitrogen source. The nitrogen source can be, for example, potassium nitrate, ammonium nitrate ammonium sulfate, ammonium phosphate, ammonia, urea, and/or ammonium chloride. These nitrogen sources may be used independently or in a combination of two or more.

The method can provide oxygenation to the growing culture. One embodiment utilizes slow motion of air to remove low-oxygen containing air and introduce oxygenated air. In the case of submerged fermentation, the oxygenated air may be ambient air supplemented daily through mechanisms including impellers for mechanical agitation of liquid, and air spargers for supplying bubbles of gas to liquid for dissolution of oxygen into the liquid.

The method can further comprise supplementing the cultivation with a carbon source. The carbon source is typically a carbohydrate, such as glucose, sucrose, lactose, fructose, trehalose, mannose, mannitol, and/or maltose; organic acids such as acetic acid, fumaric acid, citric acid, propionic acid, malic acid, malonic acid, and/or pyruvic acid; alcohols such as ethanol, propanol, butanol, pentanol, hexanol, isobutanol, and/or glycerol; fats and oils such as soybean oil, canola oil, rice bran oil, olive oil, corn oil, sesame oil, and/or linseed oil; etc. These carbon sources may be used independently or in a combination of two or more.

In one embodiment, growth factors and trace nutrients for microorganisms are included in the medium. This is particularly preferred when growing microbes that are incapable of producing all of the vitamins they require. Inorganic nutrients, including trace elements such as iron, zinc, copper, manganese, molybdenum and/or cobalt may also be included in the medium. Furthermore, sources of vitamins, essential amino acids, and microelements can be included, for example, in the form of flours or meals, such as corn flour, or in the form of extracts, such as yeast extract, potato extract, beef extract, soybean extract, banana peel extract, and the like, or in purified forms. Amino acids such as, for example, those useful for biosynthesis of proteins, can also be included.

In one embodiment, inorganic salts may also be included. Usable inorganic salts can be potassium dihydrogen phosphate, dipotassium hydrogen phosphate, disodium hydrogen phosphate, magnesium sulfate, magnesium chloride, iron sulfate, iron chloride, manganese sulfate, manganese chloride, zinc sulfate, lead chloride, copper sulfate, calcium chloride, sodium chloride, calcium carbonate, and/or sodium carbonate. These inorganic salts may be used independently or in a combination of two or more.

In one embodiment, one or more biostimulants may also be included, meaning substances that enhance the rate of growth of a microorganism. Biostimulants may be species-specific or may enhance the rate of growth of a variety of species.

In some embodiments, the method for cultivation may further comprise adding an antimicrobial in the medium before, and/or during the cultivation process.

In certain embodiments, an antibiotic can be added to a culture at low concentrations to produce microbes that are resistant to the antibiotic. The microbes that survive exposure to the antibiotic are selected and iteratively re-cultivated in the presence of progressively higher concentrations of the antibiotic to obtain a culture that is resistant to the antibiotic. This can be performed in a laboratory setting or industrial scale using methods known in the microbiological arts. In certain embodiments, the amount of antibiotic in the culture begins at, for example, 0.0001 ppm and increases by about 0.001 to 0.1 ppm each iteration until the concentration in the culture is equal to, or about equal to, the dosage that would typically be applied to a livestock animal.

In certain embodiments, the antibiotics are those often used in livestock feed to promote growth and to help treat and prevent illness and infection in animals, such as, for example, procaine, penicillin, tetracyclines (e.g., chlortetracycline, oxytetracycline), tylosin, bacitracin, neomycin sulfate, streptomycin, erythromycin, monensin, roxarsone, salinomycin, tylosin, lincomycin, carbadox, laidlomycin, lasalocid, oleandomycin, virginamycin, and bambermycins. By producing beneficial microbes that are resistant to a particular livestock antibiotic, the microbes can be selected based on which antibiotic may be administered to the animal to treat or prevent a condition. Alternatively, an antibiotic can be selected for a livestock animal based on which beneficial microbe is being administered to the animal according to the subject methods so as not to harm the beneficial microbe.

The pH of the mixture should be suitable for the microorganism of interest. Buffers, and pH regulators, such as carbonates and phosphates, may be used to stabilize pH near a preferred value. When metal ions are present in high concentrations, use of a chelating agent in the medium may be necessary.

The microbes can be grown in planktonic form or as biofilm. In the case of biofilm, the vessel may have within it a substrate upon which the microbes can be grown in a biofilm state. The system may also have, for example, the capacity to apply stimuli (such as shear stress) that encourages and/or improves the biofilm growth characteristics.

In one embodiment, the method for cultivation of microorganisms is carried out at about 5° to about 100° C., preferably, 15 to 60° C., more preferably, 25 to 50° C. In a further embodiment, the cultivation may be carried out continuously at a constant temperature. In another embodiment, the cultivation may be subject to changing temperatures.

In one embodiment, the equipment used in the method and cultivation process is sterile. The cultivation equipment such as the reactor/vessel may be separated from, but connected to, a sterilizing unit, e.g., an autoclave. The cultivation equipment may also have a sterilizing unit that sterilizes in situ before starting the inoculation. Air can be sterilized by methods know in the art. For example, the ambient air can pass through at least one filter before being introduced into the vessel. In other embodiments, the medium may be pasteurized or, optionally, no heat at all added, where the use of low water activity and low pH may be exploited to control undesirable bacterial growth.

In one embodiment, the subject invention further provides a method for producing microbial metabolites such as, for example, biosurfactants, enzymes, proteins, ethanol, lactic acid, beta-glucan, peptides, metabolic intermediates, polyunsaturated fatty acid, and lipids, by cultivating a microbe strain of the subject invention under conditions appropriate for growth and metabolite production; and, optionally, purifying the metabolite. The metabolite content produced by the method can be, for example, at least 20%, 30%, 40%, 50%, 60%, 70%, 80%, or 90%.

The biomass content of the fermentation medium may be, for example, from 5 g/l to 180 g/l or more, or from 10 g/l to 150 g/l. The cell concentration may be, for example, at least 1×10⁹, 1×10¹⁰, 1×10¹¹, 1×10¹² or 1×10¹³ cells per gram of final product.

The microbial growth by-product produced by microorganisms of interest may be retained in the microorganisms or secreted into the growth medium. The medium may contain compounds that stabilize the activity of microbial growth by-product.

The method and equipment for cultivation of microorganisms and production of the microbial by-products can be performed in a batch, a quasi-continuous process, or a continuous process.

In one embodiment, all of the microbial cultivation composition is removed upon the completion of the cultivation (e.g., upon, for example, achieving a desired cell density, or density of a specified metabolite). In this batch procedure, an entirely new batch is initiated upon harvesting of the first batch.

In another embodiment, only a portion of the fermentation product is removed at any one time. In this embodiment, biomass with viable cells, spores, conidia, hyphae and/or mycelia remains in the vessel as an inoculant for a new cultivation batch. The composition that is removed can be a cell-free medium or contain cells, spores, or other reproductive propagules, and/or a combination of thereof. In this manner, a quasi-continuous system is created.

Advantageously, the method does not require complicated equipment or high energy consumption. The microorganisms of interest can be cultivated at small or large scale on site and utilized, even being still-mixed with their media.

### Preparation of Microbe-Based Products

A “microbe-based product,” is a product to be applied in practice to achieve a desired result. The microbe-based product can be simply a microbe-based composition harvested from a microbe cultivation process. Alternatively, a microbe-based product may comprise further ingredients that have been added. These additional ingredients can include, for example, stabilizers, buffers, carriers (e.g., water or salt solutions), added nutrients to support further microbial growth, non-nutrient growth enhancers and/or agents that facilitate tracking of the microbes and/or the composition in the environment to which it is applied. The microbe-based product may also comprise mixtures of microbe-based compositions. The microbe-based product may also comprise one or more components of a microbe-based composition that have been processed in some way such as, but not limited to, filtering, centrifugation, lysing, drying, purification and the like.

One microbe-based product of the subject invention is simply the fermentation medium containing a microorganism and/or the microbial metabolites produced by the microorganism and/or any residual nutrients. The product of fermentation may be used directly without extraction or purification. If desired, extraction and purification can be easily achieved using standard extraction and/or purification methods or techniques described in the literature.

The microorganisms in the microbe-based product may be in an active or inactive form. Furthermore, the microorganisms may be removed from the composition, and the residual culture utilized. The microbe-based products may be used without further stabilization, preservation, and storage. Advantageously, direct usage of these microbe-based products preserves a high viability of the microorganisms, reduces the possibility of contamination from foreign agents and undesirable microorganisms, and maintains the activity of the by-products of microbial growth.

The microbes and/or medium (e.g., broth or solid substrate) resulting from the microbial growth can be removed from the growth vessel and transferred via, for example, piping for immediate use.

In one embodiment, the microbe-based product is simply the growth by-products of the microorganism. For example, biosurfactants produced by a microorganism can be collected from a submerged fermentation vessel in crude form, comprising, for example about 50% pure biosurfactant in liquid broth.

In other embodiments, the microbe-based product (microbes, medium, or microbes and medium) can be placed in containers of appropriate size, taking into consideration, for example, the intended use, the contemplated method of application, the size of the fermentation vessel, and any mode of transportation from microbe growth facility to the location of use. Thus, the containers into which the microbe-based composition is placed may be, for example, from 1 gallon to 1,000 gallons or more. In other embodiments the containers are 2 gallons, 5 gallons, 25 gallons, or larger.

Upon harvesting, for example, the yeast fermentation product, from the growth vessels, further components can be added as the harvested product is placed into containers and/or piped (or otherwise transported for use). The additives can be, for example, buffers, carriers, other microbe-based compositions produced at the same or different facility, viscosity modifiers, preservatives, nutrients for microbe growth, tracking agents, solvents, biocides, other microbes and other ingredients specific for an intended use.

Other suitable additives, which may be contained in the formulations according to the invention, include substances that are customarily used for such preparations. Examples of such additives include surfactants, emulsifying agents, lubricants, buffering agents, solubility controlling agents, pH adjusting agents, preservatives, stabilizers and ultra-violet light resistant agents.

In one embodiment, the product may further comprise buffering agents including organic and amino acids or their salts. Suitable buffers include citrate, gluconate, tartarate, malate, acetate, lactate, oxalate, aspartate, malonate, glucoheptonate, pyruvate, galactarate, glucarate, tartronate, glutamate, glycine, lysine, glutamine, methionine, cysteine, arginine and a mixture thereof. Phosphoric and phosphorous acids or their salts may also be used. Synthetic buffers are suitable to be used but it is preferable to use natural buffers such as organic and amino acids or their salts listed above.

In a further embodiment, pH adjusting agents include potassium hydroxide, ammonium hydroxide, potassium carbonate or bicarbonate, hydrochloric acid, nitric acid, sulfuric acid or a mixture.

In one embodiment, additional components such as an aqueous preparation of a salt, such as sodium bicarbonate or carbonate, sodium sulfate, sodium phosphate, or sodium biphosphate, can be included in the formulation.

Advantageously, in accordance with the subject invention, the microbe-based product may comprise broth in which the microbes were grown. The product may be, for example, at least, by weight, 1%, 5%, 10%, 25%, 50%, 75%, or 100% broth. The amount of biomass in the product, by weight, may be, for example, anywhere from 0% to 100% inclusive of all percentages therebetween.

Optionally, the product can be stored prior to use. The storage time is preferably short. Thus, the storage time may be less than 60 days, 45 days, 30 days, 20 days, 15 days, 10 days, 7 days, 5 days, 3 days, 2 days, 1 day, or 12 hours. In a preferred embodiment, if live cells are present in the product, the product is stored at a cool temperature such as, for example, less than 20° C., 150 C, 10° C., or 5° C. On the other hand, a biosurfactant composition can typically be stored at ambient temperatures.

### Methods for Reducing Greenhouse Gas Emissions

The compositions according to the subject invention can be used in methods for reducing enteric methane and/or other deleterious atmospheric gases and/or precursors thereof in a livestock animal's digestive system or waste. The composition can be administered enterally and/or parenterally to the animal's digestive system

“Livestock” animals, as used herein, are “domesticated” animals, meaning species that have been influenced, bred, tamed, and/or controlled over a sustained number of generations by humans, such that a mutualistic relationship exists between the animal and the human. Particularly, livestock animals include animals raised in an agricultural or industrial setting to produce commodities such as food, fiber and labor. Types of animals included in the term livestock can include, but are not limited to, alpacas, llamas, pigs (swine), horses, mules, asses, camels, dogs, ruminants, chickens, turkeys, ducks, geese, guinea fowl, and squabs.

In certain embodiments, the livestock animals are “ruminants,” or mammals that utilize a compartmentalized stomach suited for fermenting plant-based foods prior to digestion with the help of a specialized gut microbiome. Ruminants include, for example, bovines, sheep, goats, ibex, giraffes, deer, elk, moose, caribou, reindeer, antelope, gazelle, impala, wildebeest, and some kangaroos.

In specific exemplary embodiments, the livestock animals are bovine animals, which are ruminant animals belonging to the subfamily Bovinae, of the family Bovidae. Bovine animals can include domesticated and/or wild species. Specific examples include, but are not limited to, water buffalo, anoa, tamaraw, auroch, banteng, guar, gayal, yak, kouprey, domestic meat and dairy cattle (e.g., Bos taurus, Bos indicus), ox, bullock, zebu, saola, bison, buffalo, wisent, bongo, kudu, kewwel, imbabala, kudu, nyala, sitatunga, and eland.

In certain specific embodiments, the methods comprise contacting a microbe-based composition according to the subject invention with the digestive system of a livestock animal. The composition can be administered enterally and/or parenterally to, for example, the livestock animal's digestive system. For example, the composition can be administered to the livestock animal orally, via the livestock animal's feed, pasture and/or drinking water; via endoscopy; via direct injection into, e.g., the rumen, stomach, and/or intestines; via suppository; via fecal transplant; and/or via enema.

In certain embodiments, the composition can also be applied directly to the waste to reduce GHG emissions.

Advantageously, in preferred embodiments, the methods result in a reduction of methanogenic bacteria and/or protozoa present in the livestock animal's digestive system and/or waste. In certain embodiments, the methods can also result in a reduction of methane, carbon dioxide, other deleterious atmospheric gases, and/or precursors thereof, such as nitrogen and/or ammonia (precursors of nitrous oxide), in the livestock animal's digestive system and/or waste.

As used herein, “reduction” refers to a negative alteration of at least 0.25%, 0.5%, 1%, 5%, 10%, 15%, 20%, 25%, 30%, 35%, 40%, 45%, 50%, 55%, 60%, 70%, 75%, 80%, 85%, 90%, 95%, or 100%.

In some embodiments, the desired reduction is achieved within a relatively short time period, for example, within 1 week, 2 weeks, 3 weeks or 4 weeks of the animals ingesting the composition. In some embodiments, the desired reduction is achieved within, for example, 1 month, 2 months, 3 months, 4 months, 5 months or 6 months after employing the subject methods. In some embodiments, the desired reduction is achieved within 1 year, 2 years, 3 years, 4 years, or 5 years after employing the subject methods.

In certain embodiments, the methods and compositions of the subject invention decrease methanogenesis while also increasing glucose and milk synthesis. An increase in digestive enzymes by administration of the subject composition to an animal's digestive system can lead to an increase in propionate production, which effectively regulates the pH and robs methanogens of the required components for methanogenesis. In general, ruminant fermentation results in the release of negatively charged ions into the digestive system environment, increasing the acidity of the environment. These negatively charged ions are required for methanogenesis, so a highly acidic environment encourages methanogen growth; however, the synthesis of propionate also requires negatively charged ions.

In certain embodiments, the increase in propionate production achieved through the administration of the subject composition also supports milk production in nursing animals. Propionate is absorbed through the portal vein of the liver, wherein it is used to create glucose within the liver, which supports milk synthesis.

In some embodiments, the methods can further comprise adding materials to enhance the growth of the microorganisms of the subject composition at the time of application (e.g., adding nutrients and/prebiotics). In one embodiment, the nutrient sources can include, for example, sources of magnesium, phosphate, nitrogen, potassium, selenium, calcium, sulfur, iron, copper, zinc, proteins, vitamins and/or carbon. In certain embodiments, the livestock animal can be fed a source of prebiotics, which can include, for example, dry animal fodder, straw, hay, alfalfa, grains, forage, grass, fruits, vegetables, oats, and/or crop residue.

In some embodiments, prior to applying the composition, the method comprises assessing a livestock animal, herd of livestock animals, or livestock waste storage site for local conditions, determining a preferred formulation for the composition (e.g., the type, combination and/or ratios of microorganisms and/or growth by-products) that is customized for the local conditions, and producing the composition with said preferred formulation.

The local conditions can include, for example, age, health, size and species of the animal(s); herd size; purpose for producing the animal (e.g., meat, fur, fiber, labor, milk, etc.); species within the microbial population of an animal's gut and/or waste; environmental conditions, such as amount and type of GHG emissions, current climate, and/or season/time of year; mode and/or rate of application of the composition, and others as are deemed relevant.

After assessment, a preferred formulation for the composition can be determined so that the composition can be customized for these local conditions. The composition is then cultivated, preferably at a microbe growth facility that is within 300 miles, preferably within 200 miles, even more preferably within 100 miles of the location of application (e.g., an animal or livestock production facility, or a lagoon).

In some embodiments the local conditions are assessed periodically, for example, once annually, biannually, or even monthly. In this way, the composition formula can be modified in real time as necessary to meet the needs of the changing local conditions.

In an exemplary embodiment, the daily dosage of the composition administered to each animal is about 5 mg to about 100 grams, or about 10 mg to about 50 grams, or about 15 mg to about 25 grams, or about 20 mg to about 20 grams, or about 25 mg to about 10 grams, or about 30 mg to about 5 grams, per 100 kg of animal body weight.

In certain embodiments, the methods comprise adding the composition to drinking water and/or feed as a dietary supplement. The dietary supplement can have any suitable form such as a gravy, drinking water, beverage, yogurt, powder, granule, paste, suspension, chew, morsel, liquid solution, treat, snack, pellet, pill, capsule, tablet, sachet, or any other suitable delivery form. The dietary supplement can comprise the subject microbe-based compositions, as well as optional compounds such as vitamins, minerals, probiotics, prebiotics, and antioxidants. In some embodiments, the dietary supplement may be admixed with a feed composition or with water or other diluent prior to administration to the animal.

In some embodiments, the composition is applied to a grazing field or pasture as well as to the drinking water and/or feed.

According to the methods of the subject invention, administration of the microbe-based compositions can be performed as part of a dietary regimen, which can span a period ranging from parturition through the adult life of the animal. In certain embodiments, the animal is a young or growing animal. In some embodiments, the animal is an aging animal. In other embodiments administration begins, for example, on a regular or extended regular basis, when the animal has reached more than about 30%, 40%, 50%, 60%, or 80% of its projected or anticipated lifespan.

In some embodiments, the methods of the subject invention can be utilized by a livestock producer or waste processor for reducing carbon credit usage. Thus, in certain embodiments, the subject methods can further comprise conducting measurements to assess the effect of the method on reducing the generation of carbon dioxide and/or other deleterious atmospheric gases, and/or precursors thereof (e.g., nitrogen and/or ammonia), and/or to assess the effect of the method on the control of methanogens in the livestock animal's digestive system and/or waste, using standard techniques in the art.

These measurements can be conducted according to known methods in the art (see, e.g., Storm et al. 2012, incorporated herein by reference), including, for example, gas capture and quantification, chromatography, respiration chambers (which measure the amount of methane exhaled by an individual animal), and in vitro gas production technique (where feed is fermented under controlled laboratory and microbial conditions to determine amount of methane and/or nitrous oxide is emitted per gram of dry matter). The measurements can also come in the form of testing the microbial population in an animal, for example, by sampling milk, feces, and/or stomach contents and using, for example, DNA sequencing and/or cell plating to determine the number of methanogenic microbes present therein.

Measurements can be conducted at a certain time point after application of the microbe-based composition. In some embodiments, the measurements are conducted after about 1 week or less, 2 weeks or less, 3 weeks or less, 4 weeks or less, 30 days or less, 60 days or less, 90 days or less, 120 days or less, 180 days or less, and/or 1 year or less.

Furthermore, the measurements can be repeated over time. In some embodiments, the measurements are repeated daily, weekly, monthly, bi-monthly, semi-monthly, semi-annually, and/or annually.

### Treating Livestock Waste

In certain specific embodiments, a composition according to embodiments of the subject invention is administered directly to a manure lagoon, waste pond, tailing pond, tank or other storage facility where livestock and/or food processing waste is stored and/or treated.

Advantageously, in some embodiments, the microorganisms in the composition, e.g., NRRL B-67928 and/or NRRL B-68031, can facilitate increased decomposition of manure while reducing the amount of GHG emitted therefrom, e.g., methane, carbon dioxide and/or nitrous oxide. Furthermore, in some embodiments, applying the composition to manure enhances the value of the manure as an organic fertilizer due to the ability of the microorganisms to inoculate the soil of a field or crop to which the manure is eventually applied. The microorganisms and their growth by-products can improve soil biodiversity, enhance rhizosphere properties, and enhance plant growth and health, which can lead to, for example, a reduced need for nitrogen-rich synthetic fertilizers.

In some embodiments, the lagoon or waste pond comprises other animal and/or food processing waste by-products, for example, palm oil processing waste (e.g., palm oil mill effluent), olive oil processing waste (e.g., olive press cake and olive mill wastewater), dairy processing waste (e.g., acid-whey), and slaughterhouse waste (e.g., livestock carcass remnants). These high-fat waste products produce pollution and foul odors, and can form semi-solid fat layers on top of wastewater, which encourages the growth of GHG-producing microorganisms.

In certain embodiments, the microorganisms of the subject compositions can help metabolize the fat layer and increase the decomposition rate, in addition to providing GHG-reducing benefits as described previously.

In certain embodiments, the method comprises supplementing the composition with a biosurfactant, e.g., a rhamnolipid and/or a sophorolipid, which can enhance the breakdown of fats and enhance the control of GHG-producing microorganisms.

### EXAMPLES

A greater understanding of the present invention and of its many advantages may be had from the following examples, given by way of illustration. The following examples are illustrative of some of the methods, applications, embodiments and variants of the present invention. They are not to be considered as limiting the invention. Numerous changes and modifications can be made with respect to the invention.

### Example 1—In Vitro Testing

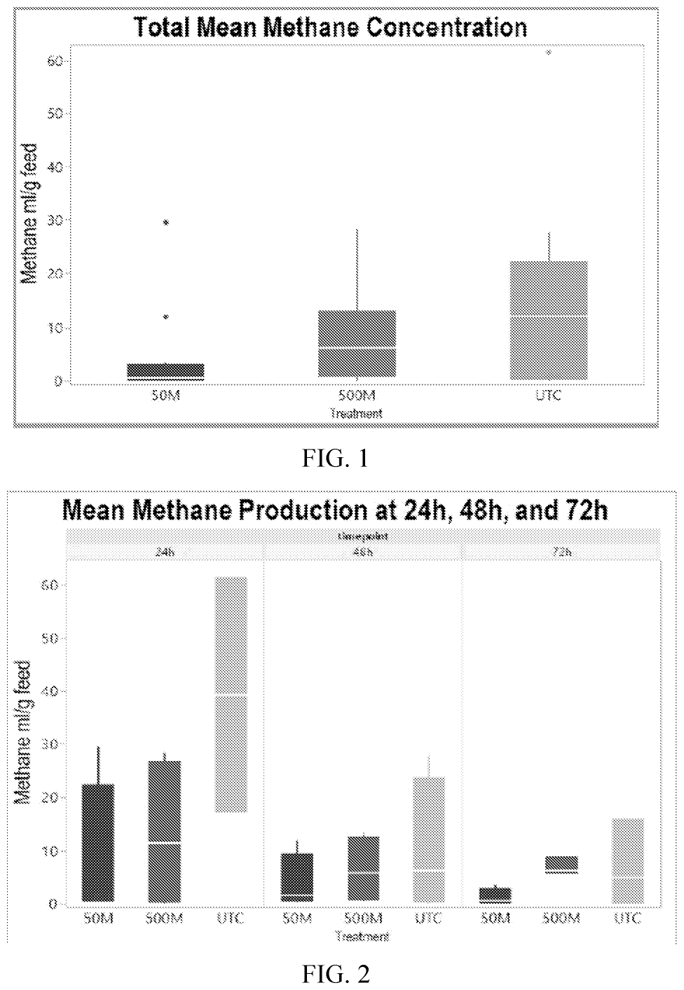

Compositions according to embodiments of the subject invention were screened for their ability to reduce enteric methane emissions in cattle. FIG. 1 shows a comparison of two dosages of the composition (50M and 500M) with an untreated control regarding the ml of methane produced per gram of feed. FIG. 2 shows the same comparison over time at 24 hours, 48 hours and 72 hours. Both doses of the composition showed reduced methane emissions over the control, with the 50M dose resulting in production of the least amount of methane over time.

### Example 2—pH Growth Testing of B4 Strain

The B4 strain was spread on Tryptic Soy Agar (TSA) at a neutral (6.8) and acidic (4.8) pH to look for differences in growth, with the goal of determining how it would behave in the various pH environments within a cow's digestive system.

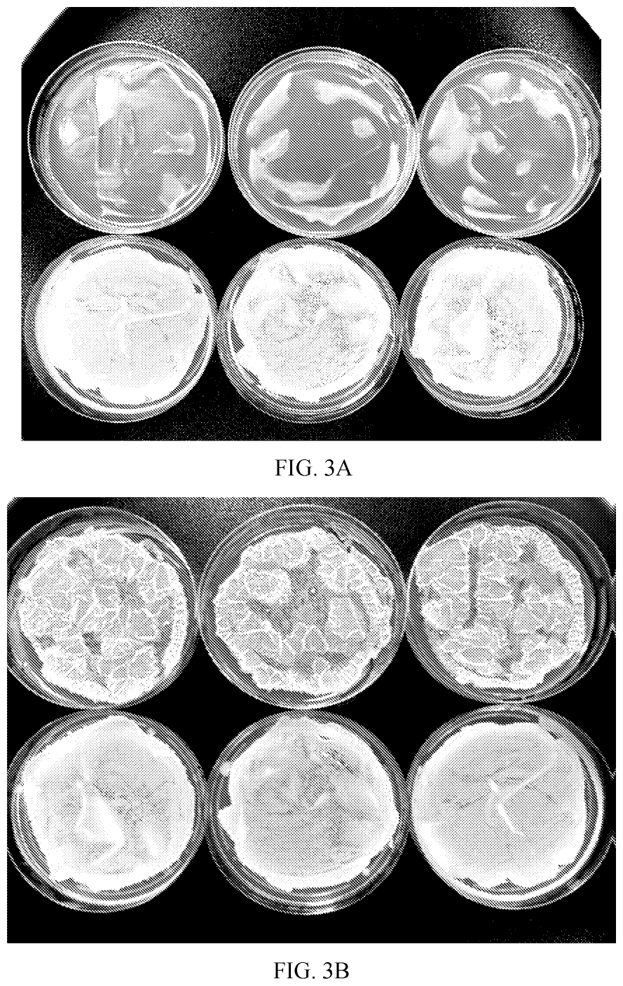

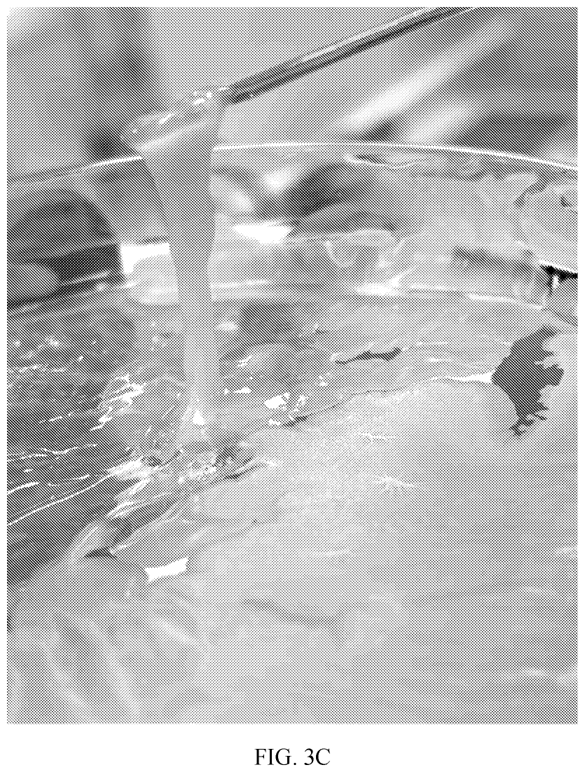

Growth at neutral pH (6.8) was faster within 24 hours compared to the acidic plates, but at 48 hours, the growth on pH 4.8 agar was equal or greater. FIGS. 3A-3B. Additionally, a significant amount of exopolysaccharide (EPS) was produced when grown on an acidic agar. FIG. 3C. This is a result of environmental stress.

The dried B4 spores were also added in sterile PBS adjusted to pH 2.8 and left for 24 hours. The same was then plated on neutral (6.8) TSA plates. A lawn of growth was present, but no EPS was produced (similar to pH 6.8 plates from FIG. 3). This shows that dried spores exposed to an overall harsh environment were still intact and viable. Overall, the pH of the growing media/environment influences EPS production.

### Example 3—EPS

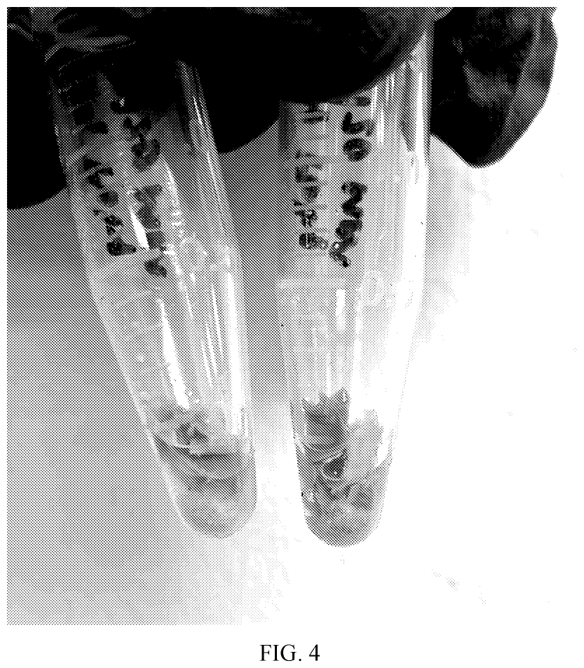

B4 was grown in a liquid medium at pH 4.8 specifically for the production of EPS. The assumed EPS was isolated out of the culture and purified. FIG. 4.

FTIR analysis confirmed the purified sample to be an EPS and HPLC analysis confirmed the existence of a large peak denoting a sugar oligomer. A C═O bond was also observed via UV absorption.

Additionally, and surprisingly, when the culture was processed for EPS extraction, there was a purple stripe present in the cell pellet. When the EPS purification was completed, the sample was a pink color, suggesting the presence of pulcherrimin or pulcherriminic acid. FIG. 4.

### Example 4—Enzyme Assay Testing—Amylase

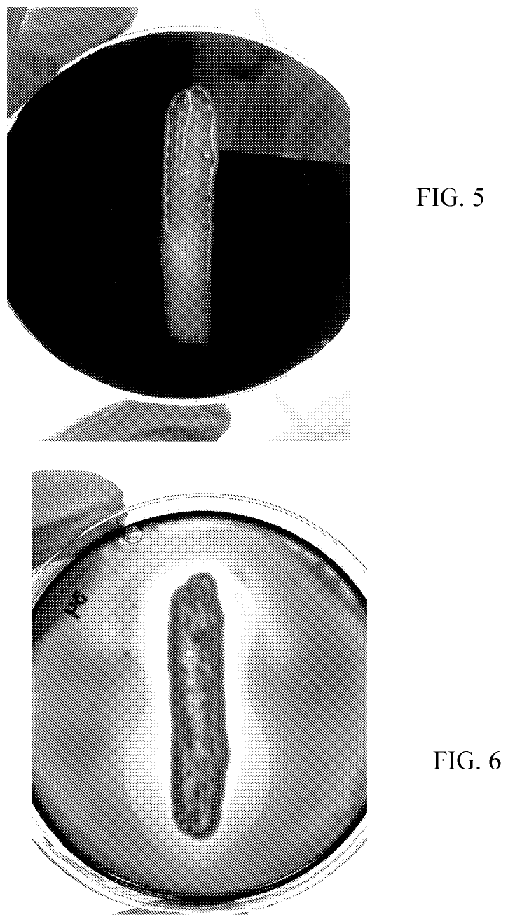

Amylase is an enzyme that hydrolyzes the glycosidic bonds in starch molecules by converting complex carbohydrates to simple sugars. Agar, a starch, was inoculated with B4 and incubated for growth. FIG. 5. After running the plate assay, an orange color around the bacterial growth was observed, indicating the breakdown of starch. This is a positive amylase test.

### Example 5—Enzyme Assay Testing—Cellulase

Cellulases are enzymes that convert cellulose to glucose. B4 was tested for cellulase activity using carboxymethylcellulose agar (CMCA) media plates.

B4 was grown in tryptic soy broth with and without cellobiose added. After 48 hours of growth, the liquid culture was streaked onto CMCA plates. After inoculation, and once growth was present on the agar, an iodine solution was introduced to the plates. A yellow zone of clearing around the bacterial growth indicates the breakdown of cellulose and the presence of cellulase enzymes. FIG. 6.

### Example 6—Detection of Siderophores

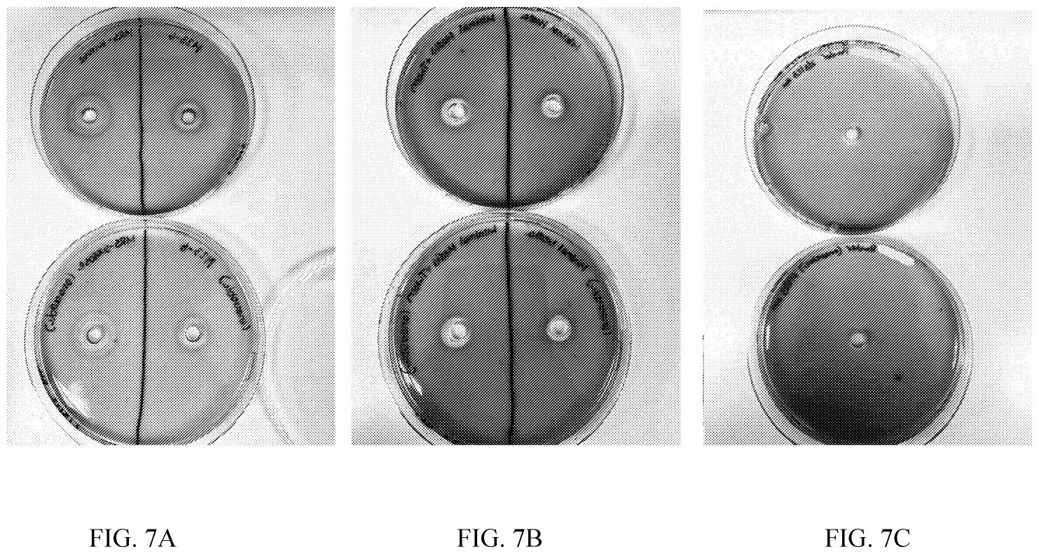

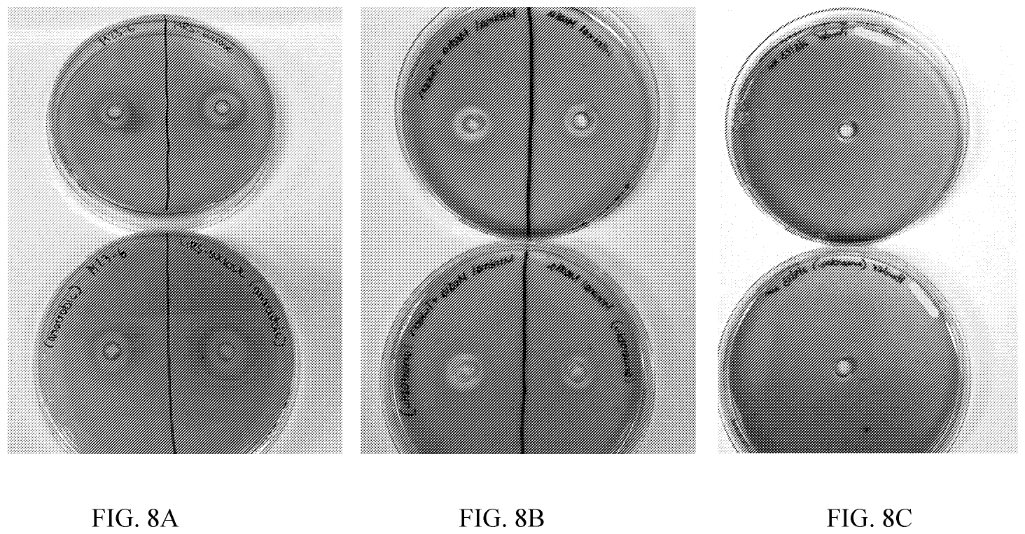

Chrome azurol S (CAS) assay was used for the detection of siderophores from B4 cultures and dried B4 spores. The tests were run in aerobic and anaerobic environments on different growth media: MRS-sucrose, M23-6, minimal media with Tween, and minimal media without Tween. The plates were observed at 6 hours (FIGS. 7A-7C) and 24 hours (FIGS. 8A-8C).

Siderophores scavenge iron from an Fe-CAS-hexadecyltrimethylammonium bromide complex, and the release of the CAS dye results in a color change from blue to orange. Any observable color change on CAS agar plates indicates a qualitative detection of siderophores.

All B4 media cultures tested were positive for siderophore production and activity. MRS-sucrose (the richest media) produced the strongest activity. There were no differences in siderophore activity between aerobic and anaerobic conditions with the four media cultures.

Dried B4 spores produced less siderophore activity compared to the B4 cultures, which may due to spore dormancy. The dried spores performed better in aerobic conditions.

### Example 7—Iron Activity Assay

To understand how B4 interacts with iron, and to determine how iron activity differs between culture media, an iron assay kit (Sigma-Aldrich) was used to determine the concentration of ferrous (Fe²⁺), ferric (Fe³⁺) and total iron present in different B4 cultures.

Iron is released from the sample by the addition of an acidic buffer. Released iron is reacted with chromagen, resulting in a colorimetric (593 nm) product that is proportional to the iron present.

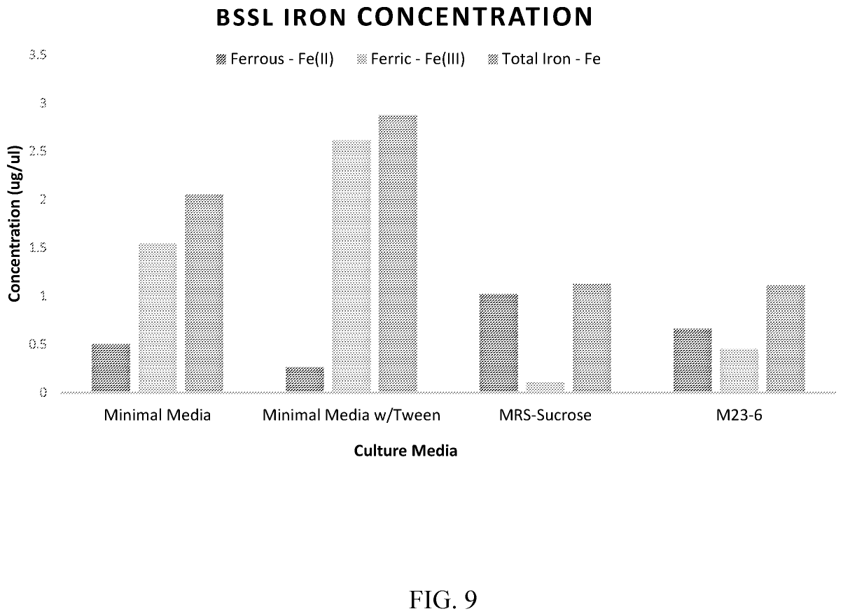

The results in FIG. 9 correlate with the siderophore results reported in FIGS. 7-8, meaning higher siderophore activity correlates with lower total iron levels. For example, MRS-sucrose media is the richest media with the highest siderophore activity and the lowest total iron levels. Additionally, the minimal media used is designed for increased production of pulcherrimin production, which is a ferric chelate. Greater levels of pulcherrimin in the culture should increase ferric and total iron concentrations.

### REFERENCES

Government of Western Australia. (2018). “Carbon farming: reducing methane emissions from cattle using feed additives.” https://www.agric.wa.gov.au/climate-change/carbon-farming-reducing-methane-emissions-cattle-using-feed-additives. (“Carbon Farming 2018”).
Gerber, P. J., et al. (2013). “Tackling climate change through livestock—A global assessment of emissions and mitigation opportunities.” Food and Agriculture Organization of the United Nations, Rome. Viewed Apr. 5, 2019. http://www.fao.org/3/i3437e/i3437e.pdf. (“Gerber et al. 2013”).
Ishler, V. A., (2016). “Carbon, Methane Emissions and the Diary Cow.” Penn State College of Agricultural Sciences. https://extension.psu.edu/carbon-methane-emissions-and-the-dairy-cow. (“Ishler 2016”).
Patra, A., Park, T., Kim, M. et al. (2017). Rumen methanogens and mitigation of methane emission by anti-methanogenic compounds and substances. J Animal Sci Biotechnol 8, 13. https://doi.org/10.1186/s40104-017-0145-9 (“Patra et al. 2017”).
Pidwirny, M. (2006). “The Carbon Cycle”. Fundamentals of Physical Geography, 2nd Edition. Viewed Oct. 1, 2018. http://www.physicalgeography.net/fundamentals/9r.html. (“Pidwirny 2006”).
Storm, I. et al. (2012). “Methods for Measuring and Estimating Methane Emission from Ruminants.” Animals (Basel). June 2(2): 160-183. doi: 10.3390/ani2020160. (“Storm et al. 2012”).
United States Environmental Protection Agency. (2016). “Climate Change Indicators in the United States.” https://www.epa.gov/sites/production/files/2016-08/documents/climate_indicators_2016.pdf. (“EPA Report 2016”).
United States Environmental Protection Agency. (2016). “Overview of Greenhouse Gases.” Greenhouse Gas Emissions. https://www.epa.gov/ghgemissions/overview-greenhouse-gases. (“Greenhouse Gas Emissions 2016”).

1. A method for reducing methane produced in a livestock animal's digestive system and/or waste, wherein the method comprises contacting, with the livestock animal's digestive system and/or waste, a composition comprising an iron-capturing Bacillus spp. bacterium and/or an iron-capturing growth product thereof.

2-3. (canceled)

4. The method of claim 1, wherein the microorganism is Bacillus subtilis B4 NRRL B-68031 or Bacillus amyloliquefaciens NRRL B-67928.

5. The method of claim 1, wherein the microorganism is administered in spore form, but grows in biofilm form upon contact with the livestock animal's digestive system.

6. The method of claim 1, comprising contacting the digestive system and/or waste with a further iron-capturing agent selected from pulcherrimin, pulcherriminic acid, citrate, citric acid, EDTA (Ethylenediaminetetraacetic acid), ferric EDTA, DTPA (Diethylenetriaminepentaacetic acid), EDDHA (Ethylenediamine di(o-hydroxyphenylacetic acid), N,N-dihydroxy-N,N′-diisopropylhexanediamide (DPH), 2,3-dihydroxybenzoic acid, azotochelin, transferrin, enterobactin, pyoverdine, protochelin, pyochelin, bacillibactin, vibriobactin, vibrioferrin azotobactin, aminochelin, yersiniabactin, agrobactin, staphyloferrin, ferrichrome, defarasirox, deferiprone, desferrioxamine, fusarinine, chrysobactin, achromobactin, ornibactin, rhodotorulic acid, lysine, glutamic acid, gluconic acid, iron oxyhydroxide minerals, ferrihydrite, magnetite, hematite, geothite, sideritehydroxamate, catecholates, salicylates, carboxylates, mugineic acid, ferulic acid, caffeic acid, and/or nicotianamine.

7. The method of claim 1, wherein the composition is administered directly to the digestive system orally, via endoscopy, or via injection into the stomach, rumen and/or intestines.

8. (canceled)

9. The method of claim 1, further comprising administering a prebiotic selected from dry animal fodder, straw, hay, alfalfa, grains, forage, grass, fruits, vegetables, oats, and crop residue.

10. The method of claim 1, further comprising administering a germination enhancer selected from L-alanine, L-leucine or manganese.

11. The method of claim 1, further comprising administering one or more of the following components: nitrates, seaweed (Asparagopsis taxiformis); kelp; 3-nitrooxypropanol; anthraquinones; ionophores selected from monensin and lasalocid; polyphenols selected from saponins and tannins; Yucca schidigera extract (steroidal saponin-producer); Quillaja saponaria extract (triterpenoid saponin-producing plant species); organosulfurs; garlic extract; flavonoids selected from quercetin, rutin, kaempferol, naringin, and anthocyanidins; bioflavonoids isolated from green citrus fruits, rose hips and/or black currants; carboxylic acid; and terpenes selected from d-limonene, pinene and citrus extracts.

12-14. (canceled)

15. The method of claim 1, wherein the composition is applied to drinking water and/or to feed that the livestock animal ingests.

16. The method of claim 1, further comprising assessing the effect of the method on the reduction of enteric deleterious atmospheric gas emissions and/or precursors thereof in the livestock animal's digestive system and/or waste.

17. The method of claim 1, further comprising assessing the effect of the method on the control of methanogenic bacteria in the livestock animal's digestive system and/or waste.

18. The method of claim 1, used for reducing the number of carbon credits used by an operator involved in livestock production.

19. A composition for reducing enteric methane in a livestock animal's digestives system, the composition comprising an iron-capturing Bacillus spp. bacterium and/or an iron-capturing growth by-product thereof, wherein the microorganism is Bacillus subtilis B4 NRRL B-68031 or Bacillus amyloliquefaciens NRRL B-67928.

20-22. (canceled)

23. The composition of claim 19, wherein the microorganism is in spore form or biofilm form.

24. The composition of claim 19, wherein the composition comprises a further iron-capturing agent selected from pulcherrimin, pulcherriminic acid, citrate, citric acid, EDTA (Ethylenediaminetetraacetic acid), ferric EDTA, DTPA (Diethylenetriaminepentaacetic acid), EDDHA (Ethylenediamine di(o-hydroxyphenylacetic acid), N,N-dihydroxy-N,N′-diisopropylhexanediamide (DPH), 2,3-dihydroxybenzoic acid, azotochelin, transferrin, enterobactin, pyoverdine, protochelin, pyochelin, bacillibactin, vibriobactin, vibrioferrin azotobactin, aminochelin, yersiniabactin, agrobactin, staphyloferrin, ferrichrome, defarasirox, deferiprone, desferrioxamine, fusarinine, chrysobactin, achromobactin, ornibactin, rhodotorulic acid, lysine, glutamic acid, gluconic acid, iron oxyhydroxide minerals, ferrihydrite, magnetite, hematite, geothite, sideritehydroxamate, catecholates, salicylates, carboxylates, mugineic acid, ferulic acid, caffeic acid, and nicotianamine.

25. (canceled)

26. The composition of claim 19, further comprising a germination enhancer, wherein the germination enhancer is L-alanine, L-leucine or manganese.

27. The composition of claim 19, further comprising one or more of the following components: seaweed (Asparagopsis taxiformis); kelp; 3-nitrooxypropanol; anthraquinones; ionophores selected from monensin and lasalocid; polyphenols selected from saponins and tannins; Yucca schidigera extract (steroidal saponin-producer); Quillaja saponaria extract (triterpenoid saponin-producing plant species); organosulfurs; garlic extract; flavonoids selected from quercetin, rutin, kaempferol, naringin, and anthocyanidins; bioflavonoids isolated from green citrus fruits, rose hips and/or black currants; carboxylic acid; and terpenes selected from d-limonene, pinene and citrus extracts.

28-29. (canceled)

30. The composition of claim 19, wherein the composition comprises a microbial growth by-product selected from biosurfactants, enzymes, organic acids, fatty acids, amino acids, proteins, peptides, alcohols, polyketides, natural antibiotics, aldehydes, amines, sterols and vitamins.

31. (canceled)

32. The composition of claim 19, further comprising a saturated long chain fatty acid selected from stearic acid, palmitic acid and myristic acid.

33. (canceled)
